(19) United States
(12) Patent Application Publication
(10) Pub. No.: US 2025/0034428 A1
(43) Pub. Date: Jan. 30, 2025

(54) COMPOUND, COMPOSITION, SURFACE TREATMENT AGENT, METHOD OF PRODUCING ARTICLE, AND ARTICLE

(71) Applicant: AGC Inc., Tokyo (JP)

(72) Inventors: Hiroyuki HARA, Tokyo (JP); Koki WATANABE, Tokyo (JP); Motoshi AOYAMA, Tokyo (JP)

(73) Assignee: AGC Inc., Tokyo (JP)

(21) Appl. No.: 18/892,759

(22) Filed: Sep. 23, 2024

Related U.S. Application Data
(63) Continuation of application No. PCT/JP2023/008167, filed on Mar. 3, 2023.

(30) Foreign Application Priority Data
Mar. 24, 2022 (JP) 2022-049074

(51) Int. Cl. C09D 183/06; C07C 11/02; C07C 57/03; C07C 69/587; C07F 7/18; C08G 77/18; C08G 77/26; C09D 5/16; C09D 183/08; G02B 1/04

(52) U.S. Cl. CPC C09D 183/06; C07C 11/02; C07C 57/03; C07C 69/587; C07F 7/1804; C08G 77/18; C08G 77/26; C09D 5/1675; C09D 183/08; G02B 1/04

(57) ABSTRACT

A compound containing a linear alkyl group having 19 or more carbon atoms or a linear alkylene group having 19 or more carbon atoms, and a reactive silyl group linked to the linear alkyl group having 19 or more carbon atoms or the linear alkylene group having 19 or more carbon atoms, directly or via a polyvalent linking group, a composition and a surface treatment agent including the compound, and an article and a method of producing an article using the surface treatment agent.

### CROSS-REFERENCE TO RELATED APPLICATION

This application is a Continuation of International Application No. PCT/JP2023/008167, filed Mar. 3, 2023, which claims priority to Japanese Patent Application No. 2022-049074, filed Mar. 24, 2022. Each of the above applications is hereby expressly incorporated by reference, in its entirety, into the present application.

### TECHNICAL FIELD

The present disclosure relates to a compound, a composition, a surface treatment agent, a method of producing an article, and an article.

### BACKGROUND ART

In recent years, in order to improve the properties of articles, such as their appearance and visibility, there is a need for a technique for making the surface of articles less susceptible to fingerprints and a technique for making it easier to remove stains and the like. As a specific method, a method of performing a surface treatment on a surface of an article using a surface treatment agent is known. For example, Patent Literature 1 describes a surface treatment agent containing n-octadecyltrimethoxysilane.

### CITATION LIST

### Patent Literature

Patent Literature 1: WO 2008/016029 A1

### SUMMARY OF INVENTION

### Technical Problem

On the other hand, there is a need for further improvements in surface treatment agents from the viewpoints of water repellency, abrasion resistance, and the like. In view of such circumstances, the present disclosure relates to a compound capable of imparting favorable water repellency and abrasion resistance to a substrate, a composition, and a surface treatment agent containing the compound, and an article and a method of producing an article using the surface treatment agent.

### Solution to Problem

Means for solving the foregoing problem include the following embodiments.
<1> A compound containing: a linear alkyl group having 19 or more carbon atoms or a linear alkylene group having 19 or more carbon atoms; and a reactive silyl group linked to the linear alkyl group having 19 or more carbon atoms or the linear alkylene group having 19 or more carbon atoms, directly or via a polyvalent linking group.
<2> The compound according to <1>, wherein a number of carbon atoms of the linear alkyl group having 19 or more carbon atoms or the linear alkylene group having 19 or more carbon atoms is from 19 to 30.
<3> The compound according to <1> or <2>, wherein the reactive silyl group contains an alkoxysilyl group.
<4> A compound represented by the following Formula (1):

(A)ⱼY¹[—Si(R)ₙL₃₋ₙ]_{g} (1)
wherein, in the Formula (1),
A is CH₃(CH₂)ₘ₁— or [L₃₋ₙ(R)ₙSi—]ₖY²—(CH₂)ₘ₂—, wherein m1 is an integer of 18 or more, m2 is an integer of 19 or more, Y² is a single bond or a (k+1)-valent linking group, k is an integer of 1 or more, and definitions of R, L, and n are the same as definitions of R, L, and n in the Formula (1), respectively,
Y¹ is a single bond or a (j+g)-valent linking group,
each R independently represents a monovalent hydrocarbon group,
each L independently represents a hydrolyzable group or a hydroxyl group,
n is an integer from 0 to 2,
j is an integer of 1 or more, and
g is an integer of 1 or more.
<5> The compound according to <4>, wherein, in the Formula (1), m1 is from 18 to 29 and m2 is from 19 to 30.
<6> The compound according to <4> or <5>, wherein at least one L is an alkoxy group in the Formula (1).
<7> The compound according to any one of <4> to <6>, wherein: in the Formula (1),
   A is CH₃(CH₂)ₘ₁—, and g is an integer of 2 or more, or
   A is [L₃₋ₙ(R)ₙSi—]ₖY²—(CH₂)ₘ₂—, and each of g and k is independently an integer of 2 or more.
<8> A compound represented by the following Formula (1-1) or (1-2):

[CH₃(CH₂)ₘ₁—]ⱼ₁Y^{10a}(B)_{g1} (1-1)

(B)ₖ₂Y^{12a}—(CH₂)ₘ₂—Y^{11a}(B)_{g2} (1-2)
wherein:
in the Formula (1-1),
Y^{10a} is a single bond or a (j1+g1)-valent linking group,
each B independently represents -Q^{L}-CH═CH₂, wherein Q^{L} is a single bond or a divalent linking group,
m1 is an integer of 18 or more,
j1 is an integer of 1 or more, and
g1 is an integer of 1 or more,
in the Formula (1-2),
Y^{11a} is a single bond or a (g2+1)-valent linking group,
Y^{12a} is a single bond or a (k2+1)-valent linking group,
each B independently represents -Q^{L}-CH═CH₂, wherein Q^{L} is a single bond or a divalent linking group,
m2 is an integer of 19 or more,
k2 is an integer of 1 or more, and
g2 is an integer of 1 or more.
<9> A composition containing the compound according to any one of <1> to <7> and a liquid medium.
<10> A surface treatment agent containing the compound according to any one of <1> to <7>.
<11> The surface treatment agent according to <10>, further containing a liquid medium.
<12> A method of producing an article, the method including: subjecting a substrate to a surface treatment with the surface treatment agent according to <10> or <11> to produce an article having a surface treatment layer formed on the substrate.
<13> An article including: a substrate; and a surface treatment layer disposed on the substrate and formed by surface treatment with the surface treatment agent according to <10>.
<14> The article according to <13>, wherein the article is an optical member.
<15> The article according to <13>, wherein the article is a display or a touch panel.

### Advantageous Effects of Invention

The present disclosure provides a compound capable of imparting favorable water repellency and abrasion resistance to a substrate, a composition and a surface treatment agent containing the compound, and an article and a method of producing an article using the surface treatment agent.

### DESCRIPTION OF EMBODIMENTS

Hereinafter, forms for carrying out the embodiments according to the present disclosure will be described in detail. However, the embodiments according to the present disclosure are not limited to the following embodiments. In the following embodiments, the components (including element steps and the like) are not essential unless otherwise specified. The same applies to numerical values and ranges thereof, and the embodiments according to the present disclosure are not limited thereby.

In the present disclosure, the term “step” includes not only a step independent of other steps but also a step that cannot be clearly distinguished from other steps as long as the purpose of the step is achieved.

In the present disclosure, numerical ranges indicated using “to” includes the numerical values described before and after “to” as the minimum value and the maximum value, respectively.

In the present disclosure, each component may contain plural corresponding substances. In a case in which plural substances corresponding to a component are present in the composition, the content rate or amount of the component means the total content rate or amount of the plural substances present in the composition unless otherwise specified.

In the present disclosure, the term “layer” or “film” encompasses, when the area of the layer or the film is observed, not only a case in which the film is formed in the entire area, but also a case in which the film is formed only in part of the area.

The “surface treatment layer” means a layer formed on the surface of a substrate by surface treatment.

In the present disclosure, when a compound or a group is represented by a specific Formula (X), the compound and the group represented by the Formula (X) may be referred to as “Compound (X)” or “Compound X” and “Group (X)” or “Group X”, respectively.

In the present disclosure, a (poly)oxyalkylene group means an oxyalkylene group or a polyoxyalkylene group.

### [Compound]

The compound according to the present disclosure contains a linear alkyl group having 19 or more carbon atoms or a linear alkylene group having 19 or more carbon atoms, and a reactive silyl group linked to the linear alkyl group having 19 or more carbon atoms or the linear alkylene group having 19 or more carbon atoms, directly or via a polyvalent linking group.

It has been surprisingly found that, when a surface treatment of the surface of a substrate is performed using a composition containing the compound according to the present disclosure, the surface of the substrate has excellent water repellency and abrasion resistance. Although the reason for this is not clear, it is considered that excellent water repellency and abrasion resistance are exhibited by using a compound having a longer chain alkylene group than those of the compounds conventionally used for surface treatment agents.

When the compound according to the present disclosure includes a linear alkyl group having 19 or more carbon atoms, the compound according to the present disclosure has a reactive silyl group linked directly or via a polyvalent linking group at one end of the linear alkyl group. When the compound according to the present disclosure includes a linear alkylene group having 19 or more carbon atoms, the compound according to the present disclosure has a reactive silyl group at one end or both ends of the linear alkylene group, linked directly or via a polyvalent linking group, and preferably has reactive silyl groups at both ends. Hereinafter, the compound according to the present disclosure having a reactive silyl group at one end of a linear alkyl group having 19 or more carbon atoms or a linear alkylene group having 19 or more carbon atoms is also referred to as “single-ended compound”, and the compound according to the present disclosure having reactive silyl groups at both ends of a linear alkylene group having 19 or more carbon atoms is referred to as “double-ended compound”.

The linear alkyl group means an unsubstituted linear alkyl group, and the linear alkylene group means an unsubstituted linear alkylene group. The number of carbon atoms of the linear alkyl group or the linear alkylene group is 19 or more, and is preferably 19 or more, more preferably 20 or more, still more preferably 21 or more, from the viewpoint of water repellency and abrasion resistance. From the viewpoint of ease of production, the number of carbon atoms is preferably 30 or less, more preferably 28 or less, and still more preferably 26 or less. From these viewpoints, the number of carbon atoms is preferably from 19 to 30, more preferably from 19 to 28, and still more preferably from 19 to 26.

One or more of the linear alkyl group or the linear alkylene group may be contained in one molecule.

In the present disclosure, the “linear alkyl group having 19 or more carbon atoms or linear alkylene group having 19 or more carbon atoms” is bonded to a group other than —CH₂—. That is, the number of carbon atoms of the “linear alkyl group having 19 or more carbon atoms” means the number of carbon atoms up to the terminal of the linear alkyl group bonded to a group other than —CH₂—. Similarly, the number of carbon atoms of the “linear alkylene group having 19 or more carbon atoms” means the number of carbon atoms of the linear alkylene group from one terminal bonded to a group other than —CH₂— to the other terminal bonded to a group other than —CH₂—.

A reactive silyl group refers to a group in which a reactive group is bonded to an Si atom. The reactive group is preferably a hydrolyzable group or a hydroxy group.

A hydrolyzable group is a group that becomes a hydroxy group by a hydrolysis reaction. In other words, a silyl group having a hydrolyzable group, represented by Si-L, undergoes a hydrolysis reaction to generate a silanol group represented by Si—OH. The silanol group is further subject to a reaction between silanol groups to form an Si—O—Si bond. Further, the silanol group can undergo dehydration condensation reaction with a silanol group originated from an oxide present on the surface of the substrate to form an Si—O—Si bond. Examples of the hydrolyzable group include an alkoxy group, an aryloxy group, a halogen atom, an acyl group, an acyloxy group, and an isocyanato group (—NCO). The alkoxy group is preferably an alkoxy group having 1 to 4 carbon atoms. Here, the aryl group of the aryloxy group encompasses a heteroaryl group. The halogen atom is preferably a chlorine atom. The acyl group is preferably an acyl group having 1 to 6 carbon atoms. The acyloxy group is preferably an acyloxy group having 1 to 6 carbon atoms.

From the viewpoint of easy formation of a uniform film and excellent durability, the reactive silyl group is preferably an alkoxysilyl group or a trichlorosilyl group. The reactive silyl group is more preferably an alkoxysilyl group from the viewpoint of ease of handling of by-products generated in the reaction with the substrate. The alkoxysilyl group is preferably a dialkoxysilyl group or a trialkoxysilyl group, and more preferably a trialkoxysilyl group.

The number of reactive silyl groups contained in the compound according to the present disclosure is 1 or more, and is preferably from 1 to 18, more preferably from 2 to 12, and still more preferably from 2 to 8, from the viewpoint of further improving the abrasion resistance of the surface treatment layer. The number of the reactive silyl groups may be one.

The reactive silyl group is preferably a group represented by the following Formula (2).

—Si(R)ₙL₃₋ₙ (2)

In the Formula (2), each R independently represents a monovalent hydrocarbon group, each L independently represents a hydrolyzable group or a hydroxy group, and n represents an integer from 0 to 2.

When multiple Groups (2) are present in one molecule, the multiple Groups (2) may be the same as or different from each other. The multiple Groups (2) are preferably the same from the viewpoint of availability of raw materials and ease of production of the compound.

Each R independently represents a monovalent hydrocarbon group, and a monovalent saturated hydrocarbon group is preferable. The number of carbon atoms in R is preferably from 1 to 6, more preferably from 1 to 3, and still more preferably 1 or 2.

When L is a hydrolyzable group, the hydrolyzable group is preferably those described above.

Among them, L is preferably an alkoxy group (preferably an alkoxy group having 1 to 4 carbon atoms) or a halogen atom from the viewpoint of ease of production of the compound. L is preferably an alkoxy group having 1 to 4 carbon atoms from the viewpoint of less outgassing at the time of coating and more favorable storage stability of the compound. When long-term storage stability of the compound is desired, L is more preferably an ethoxy group. To make the reaction time after the coating short, L is more preferably a methoxy group. In the Formula (2), it is preferable that at least one L is any of the groups described above, and it is more preferable that all of the Ls are any of the groups described above.

n is an integer from 0 to 2, preferably 0 or 1, and more preferably 0. When the compound according to the present disclosure is used as a surface treatment agent, the presence of multiple Ls leads to stronger adhesion of the surface treatment layer to a substrate.

When n is 1 or less, the multiple Ls present in one molecule may be the same as or different from each other. The multiple Ls are preferably the same from the viewpoint of availability of raw materials and ease of production of the compound. When n is 2, the multiple Rs present in one molecule may be the same as or different from each other. The multiple Rs are preferably the same from the viewpoint of availability of raw materials and ease of production of the compound.

The polyvalent linking group in the compound according to the present disclosure may be any group that does not impair the purpose of the present disclosure, and examples thereof include a polyvalent linking group described as Y¹ in Formula (1) described below. The polyvalent linking group may have or does not need to have an organosiloxane residue. In the polyvalent linking group, the terminal bonded to the linear alkyl group having 19 or more carbon atoms or the linear alkylene group having 19 or more carbon atoms is not —CH₂—, and the other terminal may be —CH₂— or does not need to be —CH₂—.

The compound according to the present disclosure may have or does not need to have an organosiloxane residue.

The compound according to the present disclosure is preferably a compound represented by the following Formula (1) from the viewpoint of excellent water repellency and abrasion resistance.

(A)ⱼY¹[—Si(R)ₙL₃₋ₙ]_{g} (1)

In the Formula (1),

A is CH₃(CH₂)ₘ₁— or [L₃₋ₙ(R)ₙSi—]ₖY²—(CH₂)ₘ₂—, in which m1 is an integer of 18 or more, m2 is an integer of 19 or more, Y² is a single bond or a (k+1)-valent linking group, k is an integer of 1 or more, and the definitions of R, L, and n are the same as the definitions of R, L, and n in the Formula (1), respectively,
Y¹ is a single bond or a (j+g)-valent linking group,
each R independently represents a monovalent hydrocarbon group,
each L independently represents a hydrolyzable group or a hydroxyl group,
n is an integer from 0 to 2,
j is an integer of 1 or more, and
g is an integer of 1 or more.

In A, m1 is an integer of 18 or more, preferably an integer from 18 to 29, more preferably an integer from 18 to 27, and still more preferably an integer from 18 to 25.
m2 is an integer of 19 or more, preferably an integer from 19 to 30, more preferably an integer from 19 to 28, and still more preferably an integer from 19 to 26.
Y² is a single bond or a (k+1)-valent linking group. Here, in a case in which Y² is a single bond, k=1. Examples of Y² include a group containing an alkylene group that may or does not need to have an etheric oxygen atom or a divalent organopolysiloxane residue, a carbon atom, a nitrogen atom, a silicon atom, a divalent to octavalent organopolysiloxane residue, and a group in which Si(R)ₙL₃₋ₙ is removed from Formula (3-1A), Formula (3-1B), or Formulas (3-1A-1) to (3-1A-7) described later (excluding a case in which the terminal bonded to —(CH₂)ₘ₂— in Y² is —CH₂—).

In addition, Y² may be any of the Group (g2-1) to Group (g2-14) described later.
Y² may have or does not need to have an organosiloxane residue.

The definitions of R, L, and n are as defined for each symbol in the reactive silyl group.

The definition and preferable ranges of k are the same as the definition and preferable ranges of g described later.
Y¹ is a single bond or a (j+g)-valent linking group. In a case in which Y¹ is a single bond, j=1 and g=1. Examples of Y¹ include a group containing an alkylene group that may or does not need to have an etheric oxygen atom or a divalent organopolysiloxane residue, a carbon atom, a nitrogen atom, a silicon atom, a divalent to octavalent organopolysiloxane residue, and a group in which Si(R)ₙL₃₋ₙ is removed from Formula (3-1A), Formula (3-1B), or Formulas (3-1A-1) to (3-1A-7) described later (excluding a case in which the terminal bonded to CH₃(CH₂)ₘ₁— or —(CH₂)ₘ₂— in Y¹ is —CH₂—).
Y¹ may be any of the Group (g2-1) to Group (g2-14) described later.
Y¹ may have or does not need to have an organosiloxane residue.
j is an integer of 1 or more. From the viewpoint of more favorable water repellency of the surface treatment layer, j is preferably from 1 to 6. From the viewpoint of ease of production of the compound, j is more preferably 1.
g is an integer of 1 or more. In one aspect, g is preferably from 1 to 15, and more preferably from 1 to 6, from the viewpoint of abrasion resistance and fingerprint removability of the surface treatment layer. In one aspect, g is preferably 2 or more, more preferably from 2 to 4, and still more preferably 2 or 3, from the viewpoint of excellent abrasion resistance of the surface treatment layer. In one aspect, g is preferably from 1 to 3, more preferably 1 or 2, and still more preferably 1, from the viewpoint of excellent fingerprint removability of the surface treatment layer.

In one aspect, from the viewpoint of excellent abrasion resistance of the surface treatment layer, it is preferable that A is CH₃(CH₂)ₘ₁— and g is an integer of 2 or more, or A is [L₃₋ₙ(R)ₙSi—]ₖY²—(CH₂)ₘ₂— and each of g and k independently represents an integer of 2 or more.

From the viewpoint of more favorable abrasion resistance of the surface treatment layer, k+g is preferably from 2 to 25, more preferably from 4 to 18, and still more preferably from 6 to 12. On the other hand, from the viewpoint of more favorable fingerprint removability of the surface treatment layer, k+g is preferably from 2 to 6, more preferably from 2 to 4, and still more preferably 2.

In a case in which the Compound (1) has multiple A's, the multiple A's may be the same as or different from each other.

In a case in which the Compound (1) has multiple [—Si(R)ₙL₃₋ₙ], the multiple [—Si(R)ₙL₃₋ₙ] may be the same as or different from each other.

The Compound (1) is preferably a compound represented by the following Formula (3-1) or (3-2). From the viewpoint of excellent water repellency and abrasion resistance, a compound represented by the following Formula (3-1) is more preferable.

[CH₃(CH₂)ₘ₁—]ⱼ₁Y¹⁰[—Si(R)ₙL₃₋ₙ]_{g1} (3-1)

[L₃₋ₙ(R)ₙSi—]ₖ₂Y¹²—(CH₂)ₘ₂—Y¹¹[—Si(R)ₙL₃₋ₙ]_{g2} (3-2)

In the Formula (3-1), the definitions of m1, R, L, and n are the same as the respective definitions in the Formula (1). Y¹⁰ is a (j1+g1)-valent linking group, and specific examples thereof are the same as Y¹ in the Formula (1). j1 is an integer of 1 or more, and a specific example thereof is the same as j in the Formula (1). g1 is an integer of 1 or more, and a specific example thereof is the same as g in the Formula (1).

In the Formula (3-2), the definitions of m2, R, L, and n are the same as the respective definitions in the Formula (1). Y¹¹ is a (g2+1)-valent linking group, and specific examples thereof are the same as Y¹ in the Formula (1). Y¹² is a (k2+1)-valent linking group, and specific examples thereof are the same as Y² in the Formula (1). Each of k2 and g2 is an integer of 1 or more, and specific examples thereof are the same as k and g, respectively, in the Formula (1).

In a case in which the Compound (3-1) has multiple [CH₃(CH₂)ₘ₁—], the multiple [CH₃(CH₂)ₘ₁—] may be the same as or different from each other.

In a case in which the Compound (3-1) or the Compound (3-2) has multiple [—Si(R)ₙL₃₋ₙ], the multiple [—Si(R)ₙL₃₋ₙ] may be the same as or different from each other.

Each of the group represented by Y¹⁰[—Si(R)ₙL₃₋ₙ]_{g1} in the Formula (3-1) and the group represented by Y¹¹[—Si(R)ₙL₃₋ₙ]_{g2} and the group represented by Y¹²[—Si(R)ₙL₃₋ₙ]ₖ₂ in the Formula (3-2) is preferably a Group (3-1A) or a Group (3-1B).

-Q^{a}-X³¹(-Q^{b}-Si(R)ₙL₃₋ₙ)ₕ(—R³¹)ᵢ (3-1A)

-Q^{c}-[CH₂C(R³²)(-Q^{d}-Si(R)ₙL₃₋ₙ)]_{y}—R³³ (3-1B)

In the Group (3-1A) or the Group (3-1B), the terminal bonded to CH₃(CH₂)ₘ₁— or —(CH₂)ₘ₂— is not —CH₂—.

In the Formula (3-1A),
Q^{a} is a single bond or a divalent linking group,
X³¹ is a single bond, an alkylene group, a carbon atom, a nitrogen atom, a silicon atom, a divalent to octavalent organopolysiloxane residue, or a group having a (h+i+1)-valent ring,
Q^{b} is a single bond or a divalent linking group,
R³¹ is a hydrogen atom, a hydroxy group, or an alkyl group,
h is an integer of 1 or more, i is an integer of 0 or more, and
the definitions and specific examples of R, L, and n are the same as the definitions and specific examples of respective symbols in the reactive silyl group.

In the Formula (3-1B),
Q^{c} is a single bond or a divalent linking group,
R³² is a hydrogen atom or an alkyl group having 1 to 10 carbon atoms,
Q^{d} is a single bond or an alkylene group,
R³³ is a hydrogen atom or a halogen atom,
y is an integer from 1 to 10, and
the definitions and specific examples of R, L, and n are the same as the definitions and specific examples of respective symbols in the reactive silyl group.
Q^{a} is a single bond or a divalent linking group.

Examples of the divalent linking group include a divalent hydrocarbon group, a divalent heterocyclic group, —O—, —S—, —SO₂—, —N(R^{d})—, —C(O)—, —Si(R^{a})₂—, and a group in which two or more of these are combined.

The divalent hydrocarbon group may be a divalent saturated hydrocarbon group, a divalent aromatic hydrocarbon group, an alkenylene group, or an alkynylene group. The divalent saturated hydrocarbon group may be linear, branched, or cyclic, and examples thereof include an alkylene group. The number of carbon atoms in the divalent saturated hydrocarbon group is preferably from 1 to 20. The divalent aromatic hydrocarbon group is preferably one having 5 to 20 carbon atoms, and examples thereof include a phenylene group. In addition, the divalent hydrocarbon group may be an alkenylene group having 2 to 20 carbon atoms or an alkynylene group having 2 to 20 carbon atoms.
R^{a} is an alkyl group (preferably having 1 to 10 carbon atoms) or a phenyl group. R^{d} is a hydrogen atom or an alkyl group (preferably having 1 to 10 carbon atoms).

Examples of the group in which two or more of these are combined include —OC(O)—, —C(O)O—, —C(O)S—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, —C(O)N(R^{d})-alkylene group, —N(R^{d})C(O)-alkylene group, (poly)oxyalkylene group, —OC(O)-alkylene group, —C(O)O-alkylene group, —C(O)S— alkylene group, —SO₂N(R^{d})-alkylene group, and —Si(R^{a})₂-phenylene group-Si(R^{a})₂.

In one aspect, Q^{a} is preferably a single bond, —O—, —S—, —N(R^{d})—, —C(O)N(R^{d})—, —OC(O)N(R^{d})—, —C(O)O—, —OC(O)—, or —C(O)S—, and more preferably a single bond, —OC(O)N(R^{d})—, or —OC(O)—.

X³¹ is a single bond, an alkylene group, a carbon atom, a nitrogen atom, a silicon atom, a divalent to octavalent organopolysiloxane residue, or a group having a (h+i+1)-valent ring.

The alkylene group may have or does not need to have —O—, a silphenylene skeleton group, a divalent organopolysiloxane residue, or a dialkylsilylene group. The alkylene group may have multiple groups selected from the group consisting of —O—, a silphenylene skeleton group, a divalent organopolysiloxane residue, and a dialkylsilylene group.

The number of carbon atoms in the alkylene group represented by X³¹ is preferably from 1 to 20, and more preferably from 1 to 10.

Examples of the divalent to octavalent organopolysiloxane residue include a divalent organopolysiloxane residue and a (w2+1)-valent organopolysiloxane residue described later.

When X³¹ is a group having a (h+i+1)-valent ring, Q^{a}, (-Q^{b}-Si(R)ₙL₃₋ₙ), and —R³¹ (in the case of i=1 or more) are directly bonded to the atoms that constitute the ring. Here, the ring is a ring other than an organopolysiloxane ring. Hereinafter, unless otherwise specified, the ring in X³¹ refers to a ring other than an organopolysiloxane ring.

The ring in X³¹ may be any of a monocyclic ring, a fused polycyclic ring, a bridged ring, a spiro ring, or an assembled polycyclic ring, and the atoms constituting the ring may be a carbocyclic ring composed only of carbon atoms, or may be a heterocyclic ring composed of carbon atoms and hetero atom(s) having a valence of two or more. Each of the bonds between the atoms constituting the ring may be a single bond or a multiple bond. The ring may be an aromatic ring or a non-aromatic ring.

The monocyclic ring is preferably from a 4-membered ring to an 8-membered ring, and more preferably a 5-membered ring or a 6-membered ring. The fused polycyclic ring is preferably a fused polycyclic ring in which two or more rings of from 4- to 8-membered rings are fused, and more preferably a fused polycyclic ring in which 2 or 3 rings selected from a 5-membered ring and a 6-membered ring are bonded, and a fused polycyclic ring in which one or two rings selected from a 5-membered ring and a 6-membered ring and one 4-membered ring are bonded. The bridged ring is preferably a bridged ring having a 5-membered ring or a 6-membered ring as the largest ring, and the spiro ring is preferably a spiro ring having two of 4 to 6-membered rings. The assembled polycyclic ring is preferably an assembled polycyclic ring in which 2 or 3 rings selected from a 5-membered ring and a 6-membered ring are bonded via a single bond, 1 to 3 carbon atoms, or one heteroatom having a valence of 2 or 3. In the assembled polycyclic ring, any one of Q^{a}, (-Q^{b}-Si(R)L₃₋ₙ) or R³¹ (in the case of i=1 or more) is preferably bonded to each ring.

As the hetero atom constituting the ring, a nitrogen atom, an oxygen atom, and a sulfur atom are preferable, and a nitrogen atom and an oxygen atom are more preferable. The number of the heteroatoms constituting the ring is preferably 3 or less. When the number of the heteroatoms constituting the ring is two or more, these heteroatoms may be the same as or different from each other.

The ring in X³¹ is preferably one selected from the group consisting of from a 3- to 8-membered aliphatic ring, a benzene ring, from a 3- to 8-membered heterocyclic ring, a fused ring in which 2 or 3 of these rings are fused, a bridged ring having a 5-membered ring or a 6-membered ring as the largest ring, and an assembled polycyclic ring that has two or more of these rings with the linking group being a single bond, an alkylene group having 3 or less carbon atoms, an oxygen atom or a sulfur atom, from the viewpoint of ease of production of the compound and more favorable abrasion resistance, light resistance, and chemical resistance of the surface treatment layer.

Preferred rings are a benzene ring, a 5- or 6-membered aliphatic ring, a 5- or 6-membered heterocyclic ring having a nitrogen atom or an oxygen atom, and a fused ring of a 5- or 6-membered carbon ring and from a 4- to 6-membered heterocyclic ring.

Specific examples of the ring include the rings shown below, as well as a 1,3-cyclohexadiene ring, a 1,4-cyclohexadiene ring, an anthracene ring, a cyclopropane ring, decahydronaphthalene ring, a norbornene ring, a norbornadiene ring, a furan ring, a pyrrole ring, thiophene ring, a pyrazine ring, a morpholine ring, an aziridine ring, an isoquinoline ring, an oxazole ring, an isoxazole ring, a thiazole ring, an imidazole ring, a pyrazole ring, a pyran ring, a pyridazine ring, a pyrimidine ring, and an indene ring. Rings having an oxo group (═O) are also described below.

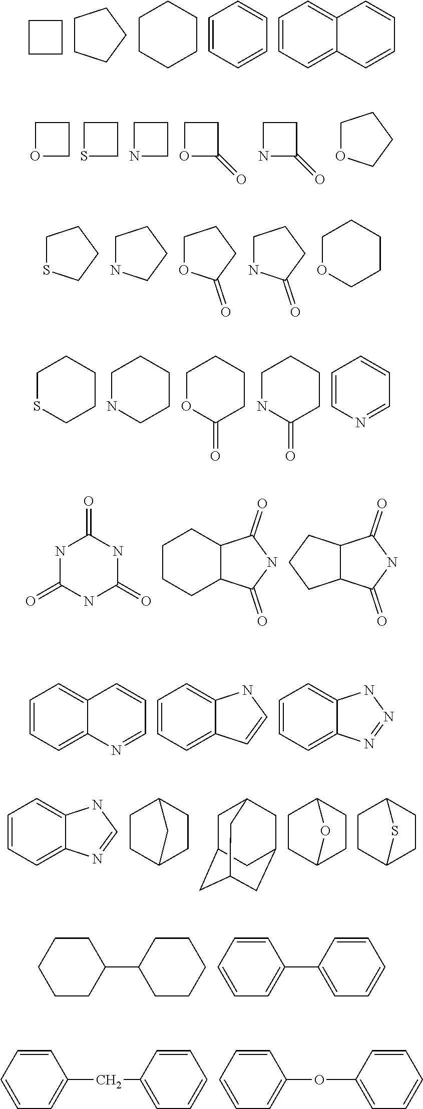

Binding sites that do not constitute the ring, of the atoms constituting the ring X³¹, are bonded to Q^{a}, (-Q^{b}-Si(R)ₙL₃₋ₙ), or R³¹ (when i=1 or more), and the remaining binding site(s), if any, are bonded to a hydrogen atom or a substituent. Examples of the substituent include a halogen atom, an alkyl group (an etheric oxygen atom may be contained between carbon atoms), a cycloalkyl group, an alkenyl group, an allyl group, an alkoxy group, and an oxo group (═O).

When one of the carbon atoms constituting the ring has two binding sites capable of bonding to Q^{a} (-Q^{b}-Si(R)ₙL₃₋ₙ), or R³¹ (when i=1 or more), any two of Q^{a}, (-Q^{b}-Si(R)ₙL₃₋ₙ), and —R³¹ may be bonded to the one carbon atom. Q^{a} and Q^{b} are preferably bonded to different ring-constituting atoms. i occurrences of R³¹s may be bonded to separate ring-constituting atoms, and two of i occurrences of R³¹s may be bonded to one ring-constituting carbon atom. There may be two or more ring-constituting carbon atoms to which two R³¹s are bonded.

Among them, from the viewpoint of improving the abrasion resistance of the surface treatment layer, X³¹ is preferably a carbon atom, a nitrogen atom, a silicon atom, quadrivalent to octavalent organopolysiloxane residue, or a group having a (h+i+1)-valent ring, and more preferably a carbon atom.

Q^{b} is a single bond or a divalent linking group.

Examples of the divalent linking group include a divalent hydrocarbon group, a divalent heterocyclic group, —O—, —S—, —SO₂—, —N(R^{d})—, —C(O)—, —Si(R^{a})₂—, and a group in which two or more of these are combined.

The divalent hydrocarbon group may be a divalent saturated hydrocarbon group, a divalent aromatic hydrocarbon group, an alkenylene group, or an alkynylene group. The divalent saturated hydrocarbon group may be linear, branched, or cyclic, and examples thereof include an alkylene group. The number of carbon atoms in the divalent saturated hydrocarbon group is preferably from 1 to 30, more preferably from 1 to 20, still more preferably from 2 to 20, and may be from 2 to 10, or from 2 to 6. Examples thereof include 2, 3, 8, 9, and 11. The divalent aromatic hydrocarbon group is preferably one having 5 to 20 carbon atoms, and examples thereof include a phenylene group. In addition, the divalent hydrocarbon group may be an alkenylene group having 2 to 20 carbon atoms or an alkynylene group having 2 to 20 carbon atoms.
R^{a} is an alkyl group (preferably having 1 to 10 carbon atoms) or a phenyl group. R^{d} is a hydrogen atom or an alkyl group (preferably having 1 to 10 carbon atoms).

Examples of the group in which two or more of these are combined include —OC(O)—, —C(O)O—, —C(O)S—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, an alkylene group having —C(O)N(R^{d})—, an alkylene group having —N(R^{d})C(O)—, an alkylene group having an etheric oxygen atom, an alkylene group having —OC(O)—, and an alkylene group having —C(O)O—, an alkylene group having —C(O)S—, an alkylene group having —SO₂N(R^{d})—, and an alkylene group-Si(R^{a})₂-phenylene group-Si(R^{a})₂.

In one aspect, Q^{c} is preferably a single bond, —O—, —S—, —N(R^{d})—, —C(O)N(R^{d})—, —OC(O)N(R^{d})—, —C(O)O—, —OC(O)—, or —C(O)S—, and more preferably a single bond, —OC(O)N(R^{d})—, or —OC(O)—.
R³¹ is a hydrogen atom, a hydroxy group, or an alkyl group.

The number of carbon atoms of the alkyl group is preferably from 1 to 5, more preferably from 1 to 3, and still more preferably 1.
h is an integer of 1 or more, and i is an integer of 0 or more.

When X³¹ is a single bond or an alkylene group, h is 1 and i is 0.

When X³¹ is a nitrogen atom, h is an integer from 1 to 2, i is an integer from 0 to 1, and h+i=2 is satisfied.

When X³¹ is a carbon atom or a silicon atom, h is an integer from 1 to 3, i is an integer from 0 to 2, and h+i=3 is satisfied.

When X³¹ is a divalent to octavalent organopolysiloxane residue, h is an integer from 1 to 7, i is an integer from 0 to 6, and h+i=1 to 7 is satisfied.

When X³¹ is a group having a (h+i+1)-valent ring, h is an integer from 1 to 7, i is an integer from 0 to 6, and h+i=1 to 7 is satisfied.

When there are two or more (-Q^{b}-Si(R)ₙL₃₋ₙ), the two or more (-Q^{b}-Si(R)ₙL₃₋ₙ) may be the same as or different from each other. When there are two or more R³¹s, the two or more (˜R³¹) may be the same as or different from each other.

Among them, i is preferably 0 from the viewpoint of improving the abrasion resistance of the surface treatment layer.
Q^{C} is a single bond or a divalent linking group.

The definition and details of the divalent linking group are the same as the definition and details in relation to Q^{a} described above.
R³² is a hydrogen atom or an alkyl group having 1 to 10 carbon atoms, and is preferably a hydrogen atom from the viewpoint of ease of production of the compound.

The alkyl group is preferably a methyl group.
Q^{d} is a single bond or an alkylene group. The number of carbon atoms in the alkylene group is preferably from 1 to 10, and more preferably from 1 to 6. Q^{d} is preferably a single bond or —CH₂— from the viewpoint of ease of production of the compound.
R³³ is a hydrogen atom or a halogen atom, and is preferably a hydrogen atom from the viewpoint of ease of production of the compound.
y is an integer from 1 to 10 and preferably an integer from 1 to 6.

Two or more [CH₂C(R³²)(-Q^{d}-Si(R)ₙL₃₋ₙ)] may be the same as or different from each other.

As the Group (3-1A), Groups (3-1A-1) to (3-1A-7) are preferable.

—(X³²)ₛ₁-Q^{b1}-Si(R)ₙL₃₋ₙ (3-1A-1)

—(X³³)ₛ₂-Q^{a2}-N[-Q^{b2}-Si(R)ₙL₃₋ₙ]₂ (3-1A-2)

-Q^{a3}-Si(R^{g})[-Q^{b3}-Si(R)ₙL₃₋ₙ]₂ (3-1A-3)

-[Q^{e}]ₛ₄-Q^{a4}-(O)ₜ₄—C[—(O)ᵤ₄-Q^{b4}-Si(R)ₙL₃₋ₙ]_{3-w1}(—R³¹)_{w1} (3-1A-4)

-Q^{a5}-Si[-Q^{b5}-Si(R)ₙL₃₋ₙ]₃ (3-1A-5)

-[Q^{e}]ᵥ-Q^{a6}-Z^{a}[-Q^{b6}-Si(R)ₙL₃₋ₙ]_{w2} (3-1A-6)

-[Q^{e}]ₛ₄-Q^{a4}-(O)ₜ₄—Z[—(O-Q^{b4})ᵤ₄-Si(R)ₙL₃₋ₙ]_{w3}(—OH)_{w4} (3-1A-7)

In the Formulas (3-1A-1) to (3-1A-7), the definitions of R, L, and n are as described above.

In the Formulas (3-1A-1) to (3-1A-7), terminals that are bonded to CH₃(CH₂)ₘ₁— or —(CH₂)ₘ₂— are not —CH₂—.

In Group (3-1A-1), X³² is —O—, —C(O)O—, —SO₂N(R^{d})—, —N(R^{d})SO₂—, —N(R^{d})C(O)—, —C(O)N(R^{d})—, —OC(O)—, —OC(O)N(R^{d})—, —S—, —C(O)S—, or —N(R^{d})— (with the proviso that N in the formula is bonded to Q^{b1})

The definition of R^{d} is as described above.
s1 is 0 or 1.
Q^{b1} is a single bond; an alkylene group; or a group in which an alkylene group that may have —O—, a silphenylene skeleton group, a divalent organopolysiloxane residue, or a dialkylsilylene group is bonded to —O—, a silphenylene skeleton group, a divalent organopolysiloxane residue, or a dialkylsilylene group; a phenylene group; or a group in which an alkylene group is bonded to a phenylene group. The number of carbon atoms in the alkylene group is preferably from 1 to 30, more preferably from 1 to 20, and still more preferably from 2 to 20.

When s1 is 0, Q^{b1} is preferably a single bond, —OCH₂CH₂CH₂—, —OCH₂CH₂OCH₂CH₂CH₂—, or —OCH₂CH₂CH₂Si(CH₃)₂OSi(CH₃)₂CH₂CH₂—.

When (X³²)ₛ₁ is —O—, Q^{b1} is preferably —CH₂CH₂CH₂— or —CH₂CH₂OCH₂CH₂CH₂—. When (X³²)ₛ₁ is —C(O)N(R^{d})—, an alkylene group having 2 to 6 carbon atoms is preferable (with the proviso that N in the formula is bonded to Q^{b}l). When Q^{b1} is any of these groups, the compound is easily produced.

Specific examples of the Group (3-1A-1) include the following groups. In the following formulas, * represents a bonding position to CH₃(CH₂)ₘ₁— or —(CH₂)ₘ₂—.

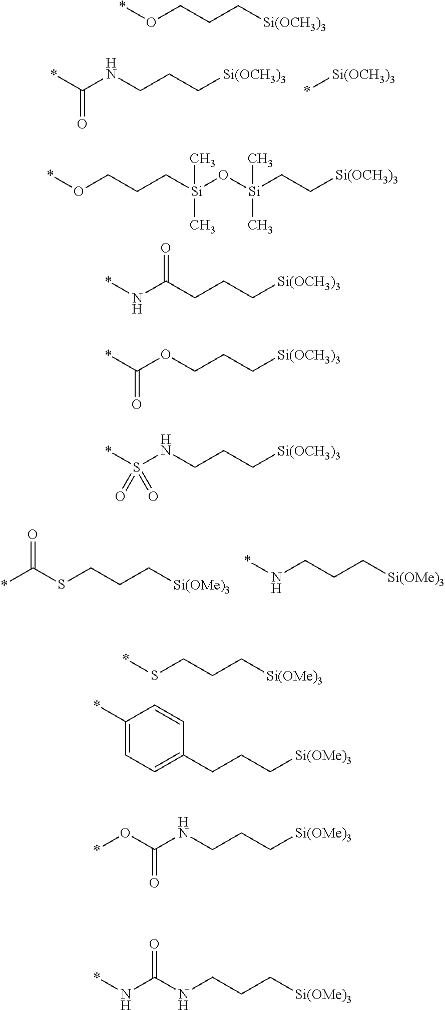

In Group (3-1A-2), X³³ is —O—, —NH—, —C(O)O—, —SO₂N(R^{d})—, —N(R^{d})SO₂—, —N(R^{d})C(O)—, or —C(O)N(R^{d})—.

The definition of R^{d} is as described above.
Q^{a2} is a single bond, an alkylene group, —C(O)—, or a group having an etheric oxygen atom, —C(O)—, —C(O)O—, —OC(O)—, —C(O)N(R^{d})—, —N(R^{d})C(O)—, —N(R^{d})C(O)N(R^{d})—, —N(R^{d}) C(O)O—, —OC(O)N(R^{d})—, —SO₂N(R^{d})—, —N(R^{d})SO₂—, —C(O)N(R^{d})—, or —NH— between carbon atoms of an alkylene group having two or more carbon atoms.

The number of carbon atoms in the alkylene group represented by Q^{a2} is preferably from 1 to 20, more preferably from 1 to 10, still more preferably from 1 to 6, and particularly preferably from 1 to 3.

The number of carbon atoms in the group having an etheric oxygen atom, —C(O)—, —C(O)O—, —OC(O)—, —C(O)N(R^{d})—, —N(R^{d})C(O)—, —N(R^{d})C(O)N(R^{d})—, —N(R^{d})C(O)O—, —OC(O)N(R^{d})—, —SO₂N(R^{d})—, —N(R^{d})SO₂—, —C(O)N(R^{d})—, or —NH— between carbon atoms of an alkylene group having two or more carbon atoms represented by Q^{a2} is preferably from 2 to 10, and more preferably from 2 to 6.
Q^{a2} is preferably —CH₂—, —CH₂CH₂—, —CH₂CH₂CH₂—, —CH₂OCH₂CH₂—, —CH₂NHCH₂CH₂—, —CH₂OC(O)CH₂CH₂—, or —C(O)— from the viewpoint of ease of production of the compound.
s2 is 0 or 1 (provided that when Q^{a2} is a single bond, s2 is 0). s2 is preferably 0 from the viewpoint of ease of production of the compound.
Q^{b2} is an alkylene group or a group having a divalent organopolysiloxane residue, an etheric oxygen atom, or —NH— between carbon atoms of an alkylene group having two or more carbon atoms.

The number of carbon atoms in the alkylene group represented by Q^{b2} is preferably from 1 to 30, more preferably from 1 to 20, still more preferably from 2 to 20, and may be from 2 to 10 or from 2 to 6. Examples thereof include 2, 3, 8, 9, and 11. The number of carbon atoms may be from 1 to 10.

The number of carbon atoms in the group having a divalent organopolysiloxane residue, an etheric oxygen atom, or —NH— between carbon atoms of an alkylene group having two or more carbon atoms represented by Q^{b2} is preferably from 2 to 10, and more preferably from 2 to 6.

Q^{b2} is preferably —CH₂CH₂CH₂— or —CH₂CH₂OCH₂CH₂CH₂— from the viewpoint of ease of production of the compound (here, the right side is bonded to Si).

Two [-Q^{b2}-Si(R)ₙL₃₋ₙ] may be the same as or different from each other.

Specific examples of the Group (3-1A-2) include the following groups. In the following formulas, * represents a bonding position to CH₃(CH₂)ₘ₁ or —(CH₂)ₘ₂—. In the formulas, a in (CH₂) a bonded to a reactive silyl group is an integer representing the number of methylene groups, and is preferably from 1 to 30, more preferably from 1 to 20, still more preferably from 2 to 20, may be from 2 to 10, or may be from 2 to 6. Examples thereof include 2, 3, 8, 9, and 11. The number of carbon atoms may be from 1 to 10. Multiple α's contained in a compound may be the same as or different from each other, and is preferably the same. For example, all of the multiple α's contained in a compound are 2, 3, 8, 9, or 11. The same applies hereinafter.

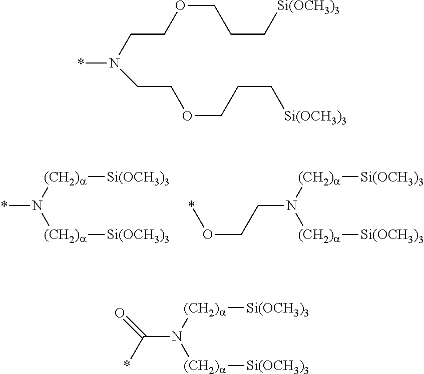

In the Group (3-1A-3), Q^{a3} is a single bond or a (poly)oxyalkylene group, and a single bond is preferable from the viewpoint of ease of production of the compound.

The number of carbon atoms in the (poly)oxyalkylene group is preferably from 1 to 20, more preferably from 1 to 10, still more preferably from 1 to 6, and particularly preferably from 1 to 3. The number of carbon atoms may be from 2 to 6.
R^{g} is a hydrogen atom, a hydroxy group, or an alkyl group. R^{g} is preferably a hydrogen atom or an alkyl group from the viewpoint of ease of production of the compound. The number of carbon atoms in the alkyl group is preferably from 1 to 10, more preferably from 1 to 4, and still more preferably, R^{g} is a methyl group.
Q^{b3} is an alkylene group or a group having an etheric oxygen atom or a divalent organopolysiloxane residue between carbon atoms of an alkylene group having two or more carbon atoms.

The number of carbon atoms in the alkylene group represented by Q^{b3} is preferably from 1 to 30, more preferably from 1 to 20, still more preferably from 2 to 20, and may be from 2 to 10 or from 2 to 6. Examples thereof include 2, 3, 8, 9, and 11. The number of carbon atoms may be from 1 to 10.

The number of carbon atoms in the group having an etheric oxygen atom or a divalent organopolysiloxane residue between carbon atoms of an alkylene group having two or more carbon atoms represented by Q^{b3} is preferably from 2 to 20, more preferably from 2 to 10, and still more preferably from 2 to 6.
Q^{b3} is preferably —CH₂CH₂—, —CH₂CH₂CH₂—, or —CH₂CH₂CH₂CH₂CH₂CH₂CH₂CH₂— from the viewpoint of ease of production of the compound.

Two [-Q^{b3}-Si(R)ₙL₃₋ₙ] may be the same as or different from each other.

Specific examples of the Group (3-1A-3) include the following group. In the following formula, * represents a bonding position to CH₃(CH₂)ₘ₁ or —(CH₂)ₘ₂—.

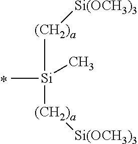

In the Group (3-1A-4), Q^{c} is —C(O)O—, —SO₂N(R^{d})—, —N(R^{d})SO₂—, —N(R^{d})C(O)—, or —C(O)N(R^{d})—.

The definition of R³¹ is as described above.
s4 is 0 or 1.
Q^{a4} is a single bond, or an alkylene group that may have an etheric oxygen atom (when s4 is 1), or a (poly)oxyalkylene group (when s4 is 0).

The number of carbon atoms in the alkylene group that may have an etheric oxygen atom or the (poly)oxyalkylene group is preferably from 1 to 20, more preferably from 1 to 10, still more preferably from 1 to 6, and particularly preferably from 1 to 3. The number of carbon atoms may be from 2 to 6.
t4 is 0 or 1 (provided that, when Q^{a4} is a single bond, Q^{a4} is 0).

As -Q^{a4}-(O)ₜ₄—, when s4 is 0, a single bond, —O—, —OCH₂—, —OCH₂CH₂O—, —OCH₂CH₂OCH₂—, or —OCH₂CH₂CH₂CH₂OCH₂— is preferable (here, the left side is bonded to CH₃(CH₂)ₘ₁— or —(CH₂)ₘ₂—), and when s4 is 1, a single bond, —CH₂—, or —CH₂CH₂— is preferable, from the viewpoint of ease of production of the compound.
Q^{b4} is an alkylene group, and the alkylene group may have or does not need to have —O—, —C(O)N(R^{d})— (the definition of R^{d} is as described above), a silphenylene skeleton group, a divalent organopolysiloxane residue, or a dialkylsilylene group.

When the alkylene group has —O— or a silphenylene skeleton group, the alkylene group preferably has —O— or the silphenylene skeleton group between carbon atoms. When the alkylene group has —C(O)N(R^{d})—, a dialkylsilylene group, or a divalent organopolysiloxane residue, the alkylene group preferably has any of these groups between carbon atoms, or at the terminal on the side at which the alkylene group is bonded to (O)ᵤ₄.

The number of carbon atoms in the alkylene group represented by Q^{b4} is preferably from 1 to 30, more preferably from 1 to 20, still more preferably from 2 to 20, and may be from 2 to 10 or from 2 to 6. Examples thereof include 2, 3, 8, 9, and 11. The foregoing range may be from 1 to 10.
u4 is 0 or 1.

As —(O)ᵤ₄-Q^{b4}-, —CH₂CH₂—, —CH₂CH₂CH₂—, —CH₂OCH₂CH₂CH₂—, —CH₂OCH₂CH₂CH₂CH₂CH₂—, —OCH₂CH₂CH₂—, —OSi(CH₃)₂CH₂CH₂CH₂—, —OSi(CH₃)₂OSi(CH₃)₂CH₂CH₂CH₂—, and —CH₂CH₂CH₂Si(CH₃)₂PhSi(CH₃)₂CH₂CH₂— are preferable from the viewpoint of ease of production of the compound (here, the right side is bonded to Si).
w1 is an integer from 0 to 2, preferably 0 or 1, and more preferably 0.

When there are two or more [—(O)ᵤ₄-Q^{b4}-Si(R)ₙL₃₋ₙ], the two or more [—(O)ᵤ₄-Q^{b4}-Si(R)ₙL₃₋ₙ] may be the same as or different from each other.

When there are two or more R³¹s, the two or more (—R³¹) may be the same as or different from each other.

Specific examples of the Group (3-1A-4) include the following groups. In the following formulas, * represents a bonding position to CH₃(CH₂)ₘ₁ or —(CH₂)ₘ₂—.

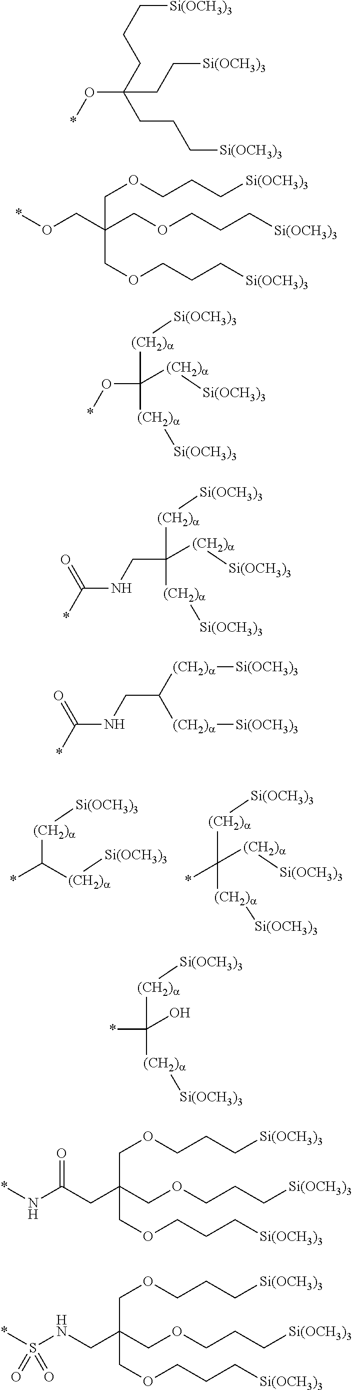

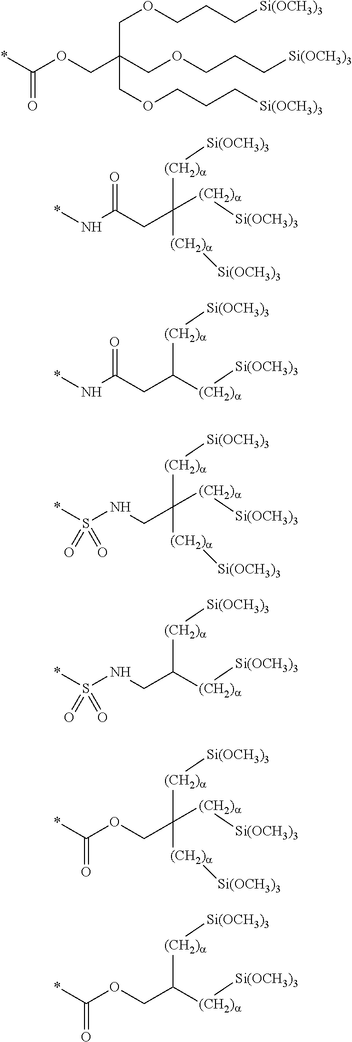

In the Group (3-1A-5), Q^{a5} is a single bond or a (poly)oxyalkylene group.

The number of carbon atoms in the (poly)oxyalkylene group is preferably from 1 to 20, more preferably from 1 to 10, still more preferably from 1 to 6, and particularly preferably from 1 to 3. The number of carbon atoms may be from 2 to 6.
Q^{a5} is preferably a single bond, —OCH₂CH₂CH₂—, or —OCH₂CH₂OCH₂CH₂CH₂— from the viewpoint of ease of production of the compound (here, the right side is bonded to Si).
Q^{b5} is an alkylene group or a group having an etheric oxygen atom or a divalent organopolysiloxane residue between carbon atoms of an alkylene group having two or more carbon atoms.

The number of carbon atoms in the alkylene group represented by Q^{b5} is preferably from 1 to 30, more preferably from 1 to 20, still more preferably from 2 to 20, and may be from 2 to 10 or from 2 to 6. Examples thereof include 2, 3, 8, 9, and 11. The number of carbon atoms may be from 1 to 10.

The number of carbon atoms in the group having an etheric oxygen atom or a divalent organopolysiloxane residue between carbon atoms of an alkylene group having two or more carbon atoms represented by Q^{b5} is preferably from 2 to 20, more preferably from 2 to 10, and still more preferably from 2 to 6.
Q^{b5} is preferably —CH₂CH₂CH₂— or —CH₂CH₂OCH₂CH₂CH₂— from the viewpoint of ease of production of the compound (here, the right side is bonded to Si(R)ₙL₃₋ₙ).

Three [-Q^{b5}-Si(R)ₙL₃₋ₙ] may be the same as or different from each other.

Specific examples of the Group (3-1A-5) include the following groups. In the following formulas, * represents a bonding position to CH₃(CH₂)ₘ₁ or —(CH₂)ₘ₂—.

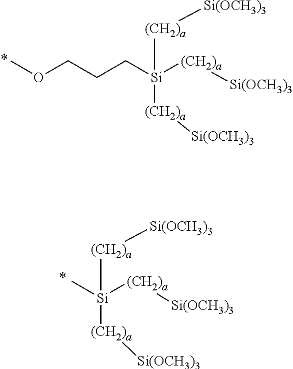

The definition of Q^{c} in the Group (3-1A-6) is as defined in the Group (3-1A-4) described above.
v is 0 or 1.
Q^{a6} is a single bond, an alkylene group that may have an etheric oxygen atom (when v is 1), or a (poly)oxyalkylene group.

The number of carbon atoms in the alkylene group that may have an etheric oxygen atom or the (poly)oxyalkylene group is preferably from 1 to 20, more preferably from 1 to 10, still more preferably from 1 to 6, and particularly preferably from 1 to 3. The number of carbon atoms may be from 2 to 6.

When v is 1, Q^{a6} is preferably —CH₂OCH₂CH₂CH₂—, —CH₂OCH₂CH₂OCH₂CH₂CH₂—, —CH₂CH₂—, or —CH₂CH₂CH₂— from the viewpoint of ease of production of the compound (here, the right side is bonded to Z^{a}).

When v is 0, Q^{a6} is preferably —OCH₂CH₂CH₂— or —OCH₂CH₂OCH₂CH₂CH₂— from the viewpoint of ease of production of the compound (here, the right side is bonded to Z^{a}).

Z^{a} is a (w2+1)-valent organopolysiloxane residue or a (w2+1)-valent group having an alkylene group between an organopolysiloxane residue and an organopolysiloxane residue.
w2 is an integer from 2 to 7.

Examples of the (w2+1)-valent organopolysiloxane residue or the (w2+1)-valent group having an alkylene group between an organopolysiloxane residue and an organopolysiloxane residue include the following groups. Here, R^{a} in the following formulas is as described above.

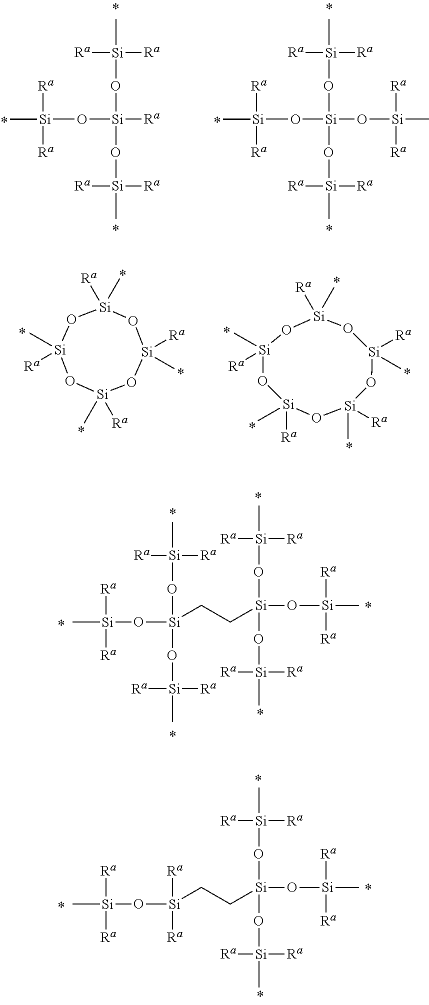

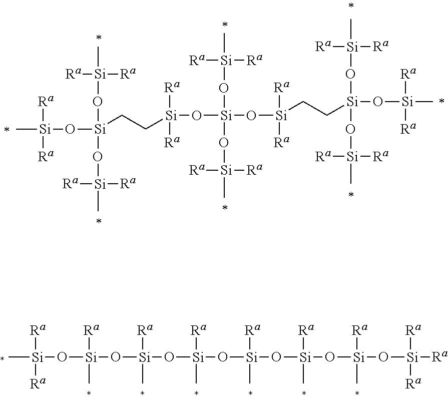

Q^{b6} is an alkylene group or a group having an etheric oxygen atom or a divalent organopolysiloxane residue between carbon atoms of an alkylene group having two or more carbon atoms.

The number of carbon atoms in the alkylene group represented by Q^{b6} is preferably from 1 to 30, more preferably from 1 to 20, still more preferably from 2 to 20, may be from 2 to 10, and may be from 2 to 6. Examples thereof include 2, 3, 8, 9, and 11. The number of carbon atoms may be from 1 to 10.

The number of carbon atoms in the group having an etheric oxygen atom or a divalent organopolysiloxane residue between carbon atoms of an alkylene group having two or more carbon atoms represented by Q^{b6} is preferably from 2 to 20, more preferably from 2 to 10, and still more preferably from 2 to 6.
Q^{b6} is preferably —CH₂CH₂— and —CH₂CH₂CH₂— from the viewpoint of ease of production of the compound.
w2 occurrences of [-Q^{b6}-Si(R)ₙ₃L₃₋ₙ] may be the same as or different from each other.

Specific examples of the Group (3-1A-6) include the following group. In the following formula, * represents a bonding position to CH₃(CH₂)ₘ₁— or —(CH₂)ₘ₂—.

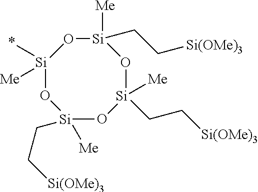

In the Group (3-1A-7), Z^{c} is a (w3+w4+1)-valent hydrocarbon group.
w3 is an integer of 4 or more.
w4 is an integer of 0 or more.

The definitions and preferred ranges of Q, s4, Q^{a4}, t4, Q^{b4}, and u4 are the same as the definitions of the respective symbols in the Group (3-1A-4).

Z° may be composed of a hydrocarbon chain, or may have an etheric oxygen atom between carbon atoms of a hydrocarbon chain, and is preferably composed of a hydrocarbon chain.

The valence of Z° is preferably from 5 to 20, more preferably from 5 to 10, still more preferably from 5 to 8, and particularly preferably 5 or 6.

The number of carbon atoms in Z° is preferably from 3 to 50, more preferably from 4 to 40, and still more preferably from 5 to 30.
w3 is preferably from 4 to 20, more preferably from 4 to 16, still more preferably from 4 to 8, and particularly preferably 4 or 5.
w4 is preferably from 0 to 10, more preferably from 0 to 8, still more preferably from 0 to 6, particularly preferably from 0 to 3, and most preferably 0 or 1.

When there are two or more [—(O-Q^{b4})ᵤ₄-Si(R)ₙL₃₋ₙ], the two or more [—(O-Q^{b4})ᵤ₄-Si(R)ₙL₃₋ₙ] may be the same as or different from each other.

Specific examples of the Group (3-1A-7) include the following groups. In the following formula, * represents a bonding position to CH₃(CH₂)ₘ₁— or —(CH₂)ₘ₂—.

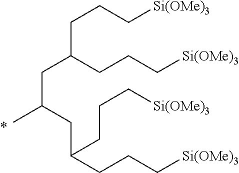

Y¹⁰ in the Formula (3-1) may be a Group (g2-1) (in which j1=d1+d3 and g1=d2+d4), a Group (g2-2) (in which j1=e1 and g1=e2), a Group (g2-3) (in which j1=1 and g1=2), a Group (g2-4) (in which j1=h1 and g1=h2), a Group (g2-5) (in which j1=i1 and g1=i2), a Group (g2-6) (in which j1=1 and g1=1), or a Group (g2-7) (in which j1=1 and g1=i3).

Each of Y¹¹ and Y¹² in the Formula (3-2) may independently be a Group (g2-1) (in which g2=d2+d4 and k2=d2+d4), a Group (g2-2) (in which g2=e2 and k2=e2), a Group (g2-3) (in which g2=2 and k2=2), a Group (g2-4) (in which g2=h2 and k2=h2), a Group (g2-5) (in which g2=i2 and k2=i2), a Group (g2-6) (in which g2=1 and k2=1), or a Group (g2-7) (in which g2=i3 and k2=i3).

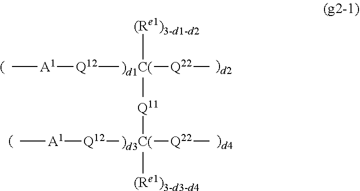

(-A¹-Q¹²-)ₑ₁C(R^{e2})₄₋ₑ₁₋ₑ₂(-Q²²-)ₑ₂ (g2-2)

-A¹-Q¹³-N(-Q²³-)₂ (g2-3)

(-A¹-Q¹⁴-)ₕ₁Z¹(-Q²⁴-)ₕ₂ (g2-4)

(-A¹-Q¹⁵-)ᵢ₁Si(R^{e3})₄₋ᵢ₁₋ᵢ₂(-Q²⁵-)ᵢ₂ (g2-5)

-A¹-Q²⁶- (g2-6)

-A¹-Q¹²-CH(-Q²²-)—Si(R^{e3})₃₋ᵢ₃(-Q²⁵-)ᵢ₃ (g2-7)

In the Formulas (g2-1) to (g2-7), the A¹ side is connected to CH₃(CH₂)ₘ₁— or —(CH₂)ₘ₂—, and the Q²², Q²³, Q²⁴, Q²⁵, or Q²⁶ side is connected to [—Si(R)ₙL₃₋ₙ].

In the Formulas (g2-1) to (g2-7), the terminals bonded to CH₃(CH₂)ₘ₁— or —(CH₂)ₘ₂— are not —(CH₂)—.

A¹ is a single bond, —C(O)NR⁶—, —C(O)—, —OC(O)O—, —NHC(O)O—, —NHC(O)NR⁶—, —O—, —NR⁶—, or —SO₂NR⁶—.
Q¹¹ is a single bond, —O—, an alkylene group, or a group having —C(O)NR⁶—, —C(O)—, —NR⁶—, or —O— between carbon atoms of an alkylene group having two or more carbon atoms.
Q¹² is a single bond, an alkylene group, or a group having —C(O)NR⁶—, —C(O)—, —NR⁶—, or —O— between carbon atoms of an alkylene group having two or more carbon atoms, and when Y¹⁰, Y¹¹ or Y¹² has two or more Q¹²s, the two or more Q¹²s may be the same as or different from each other.
Q¹³ is a single bond, an alkylene group, a group having —C(O)NR⁶—, —C(O)—, —NR⁶—, or —O— between carbon atoms of an alkylene group having two or more carbon atoms, or a group having —C(O)— at an N-side end of an alkylene group.
Q¹⁴ is Q¹² in a case in which the atom in Z¹ to which Q¹⁴ is bonded is a carbon atom, Q¹³ in a case in which the atom in Z¹ to which Q¹⁴ is bonded is a nitrogen atom, and in a case in which Y¹⁰, Y¹¹, or Y¹² has two or more Q¹⁴s, the two or more Q¹⁴s may be the same as or different from each other.
Q¹⁵ is a single bond, an alkylene group, or a group having —C(O)NR⁶—, —C(O)—, —NR⁶—, or —O— between carbon atoms of an alkylene group having two or more carbon atoms, and when Y¹⁰, Y¹¹ or Y¹² has two or more Q¹⁵s, the two or more Q¹⁵s may be the same as or different from each other.
Q²² is an alkylene group, a group having —C(O)NR⁶—, —C(O)—, —NR⁶—, or —O— between carbon atoms of an alkylene group having two or more carbon atoms, a group having —C(O)NR⁶—, —C(O)—, —NR⁶—, or —O— at a terminal of the alkylene group on a side not connected to Si, or a group having —C(O)NR⁶—, —C(O)—, —NR⁶—, or —O— between carbon atoms of an alkylene group having two or more carbon atoms and having —C(O)NR⁶—, —C(O)—, —NR⁶—, or —O— at a terminal on a side not connected to Si, and in a case in which Y¹⁰, Y¹¹, or Y¹² has two or more Q²²s, the two or more Q²²s may be the same as or different from each other.
Q²³ is an alkylene group or a group having —C(O)NR⁶—, —C(O)—, —NR⁶— or —O— between carbon atoms of an alkylene group having two or more carbon atoms, and the two Q²³s may be the same as or different from each other.
Q²⁴ is Q²² in a case in which the atom in Z¹ to which Q²⁴ is bonded is a carbon atom, Q²³ in a case in which the atom in Z¹ to which Q²⁴ is bonded is a nitrogen atom, and in a case in which Y¹⁰, Y¹¹, or Y¹² has two or more Q²⁴s, the two or more Q²⁴s may be the same as or different from each other.
Q²⁵ is an alkylene group, or a group having —C(O)NR⁶—, —C(O)—, —NR⁶—, or —O— between carbon atoms of an alkylene group having two or more carbon atoms, and in a case in which Y¹⁰, Y¹¹, or Y¹² has two or more Q²⁵s, the two or more Q²⁵s may be the same as or different from each other.
Q²⁶ is a single bond, an alkylene group, or a group having —C(O)NR⁶—, —C(O)—, —NR⁶—, or —O— between carbon atoms of an alkylene group having two or more carbon atoms.
Z¹ is a group having a (h1+h2)-valent ring structure having a carbon atom or a nitrogen atom to which Q¹⁴ is directly bonded and having a carbon atom or a nitrogen atom to which Q²⁴ is directly bonded.
R^{e1} is a hydrogen atom or an alkyl group, and in a case in which Y¹⁰, Y¹¹, or Y¹² has two or more R^{e1}s, the two or more R^{e1}s may be the same as or different from each other.
R^{e2} is a hydrogen atom, a hydroxyl group, an alkyl group, or an acyloxy group.
R^{e3} is an alkyl group.
R⁶ is a hydrogen atom, an alkyl group having 1 to 6 carbon atoms, or a phenyl group.
d1 is an integer from 0 to 3, and is preferably 1 or 2. d2 is an integer from 0 to 3, and is preferably 1 or 2. d1+d2 is an integer from 1 to 3. d3 is an integer from 0 to 3, and is preferably 0 or 1. d4 is an integer from 0 to 3, and is preferably 2 or 3. d3+d4 is an integer from 1 to 3.
d1+d3 is an integer from 1 to 5, preferably 1 or 2, in Y¹⁰, and is 1 in Y¹¹ and Y¹².
d2+d4 is an integer from 1 to 5, preferably 4 or 5, in Y¹⁰, and is an integer from 3 to 5, preferably 4 or 5, in Y¹¹ and Y¹².
e1+e2 is 3 or 4. e1 is an integer from 1 to 3, preferably 1 or 2, in Y¹⁰, and is 1 in Y¹¹ and Y¹². e2 is an integer from 1 to 3, preferably 2 or 3, in Y¹⁰, and is 2 or 3 in Y¹¹ and Y¹².
h1 is an integer of 1 or more, preferably 1 or 2, in Y¹⁰, and is 1 in Y¹¹ and Y¹². h2 is an integer of 1 or more, and is preferably 2 or 3.
i1+i2 is 3 or 4. i1 is an integer from 1 to 3, preferably 1 or 2, in Y¹⁰, and is 1 in Y¹¹ and Y¹². i2 is an integer from 1 to 3, preferably 2 or 3, in Y¹⁰, and is 2 or 3 in Y¹¹ and Y¹².
i3 is 2 or 3.

The number of carbon atoms in the alkylene group of Q¹¹, Q¹², Q¹³, Q¹⁴, Q¹⁵, Q²², Q²³, Q²⁴, Q²⁵, and Q²⁶ is preferably from 1 to 30, more preferably from 1 to 20, still more preferably from 2 to 20, may be from 2 to 10, or may be from 2 to 6, from the viewpoint of ease of production of the compound and more favorable abrasion resistance of the surface treatment layer. Examples thereof include 2, 3, 8, 9, and 11. The number of carbon atoms may be from 1 to 10, from 1 to 6, or from 1 to 4. Here, the lower limit of the number of carbon atoms of the alkylene group in the case of having a specific bond between carbon atoms is 2.

Examples of the ring structure in Z¹ include the ring structures described above, and preferred embodiments are also the same. Q¹⁴ and Q²⁴ are directly bonded to the ring structure in Z¹, and therefore, for example, there is no such case in which an alkylene group is linked to the ring structure and Q¹⁴ and Q²⁴ are linked to the alkylene group.

The number of carbon atoms in the alkyl group of R^{e1}, R^{e2}, or R^{e3} is preferably from 1 to 6, more preferably from 1 to 3, and still more preferably from 1 to 2, from the viewpoint of ease of production of the compound.

The number of carbon atoms in the alkyl group moiety of the acyloxy group of R^{e2} is preferably from 1 to 6, more preferably from 1 to 3, and still more preferably from 1 to 2, from the viewpoint of ease of production of the compound.
h1 is preferably from 1 to 6, more preferably from 1 to 4, still more preferably 1 or 2, and particularly preferably 1, from the viewpoint of ease of production of the compound and more favorable abrasion resistance of the surface treatment layer.
h2 is preferably from 2 to 6, more preferably from 2 to 4, and still more preferably 2 or 3, from the viewpoint of ease of production of the compound and more favorable abrasion resistance of the surface treatment layer.

Other embodiments of Y¹⁰ include a Group (g2-8) (in which j1=d1+d3, and g1=d2×k3+d4×k3), a Group (g2-9) (in which j1=e1 and g1=e2×k3), a Group (g2-10) (in which j1=1 and g1=2×k3), a Group (g2-11) (in which j1=h1 and g1=h2×k3), a Group (g2-12) (in which j1=i1 and g1=i2×k3), a Group (g2-13) (in which j1=1 and g1=k3), or a Group (g2-14) (in which j1=1 and g1=i3×k3).

Other embodiments of Y¹¹ and Y¹² include a Group (g2-8) (in which k2=d2×k3 and g2=d4×k3), a Group (g2-9) (in which k2=e2×k3 and g2=e2×k3), a Group (g2-10) (in which k2=2×k3 and g2=2×k3), a Group (g2-11) (in which k2=h2×k3 and g2=h2×k3), a Group (g2-12) (in which k2=i2×k3 and g2=i2×k3), a Group (g2-13) (in which k2=k3 and g2=k3), or a Group (g2-14) (in which k2=i3×k3 and g2=i3×k3).

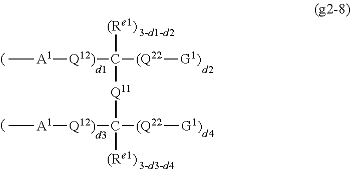

(-A¹-Q¹²-)ₑ₁C(R^{e2})₄₋ₑ₁₋ₑ₂(-Q²²-G¹)ₑ₂ (g2-9)

-A¹-Q¹³-N(-Q²³-G¹)₂ (g2-10)

(-A¹-Q¹⁴-)ₕ₁Z¹(-Q²⁴-G¹)ₕ₂ (g2-11)

(-A¹-Q¹⁵-)ᵢ₁Si(R³)₄₋ᵢ₁₋ᵢ₂(-Q²⁵-G¹)ᵢ₂ (g2-12)

-A¹-Q²⁶-G¹ (g2-13)

-A¹-Q¹²-CH(-Q²²-G¹)—Si(R^{e3})₃₋ᵢ₃(-Q²⁵-G¹)ᵢ₃ (g2-14)

In the Formulas (g2-8) to (g2-14), the A¹ side is connected to CH₃(CH₂)ₘ₁— or —(CH₂)ₘ₂—, and the G¹ side is connected to [—Si(R)ₙL₃₋ₙ]. G¹ is a Group (g3), and two or more G¹s of Y¹⁰, Y¹¹, or Y¹² may be the same as or different from each other. The symbols other than G¹ are the same as the symbols in the Formulas (g2-1) to (g2-7).

—Si(R⁸)₃₋ₖ₃(-Q³-)ₖ₃ (g3)

In the Formula (g3), the Si side is connected to Q²², Q²³, Q²⁴, Q²⁵, and Q²⁶, and the Q³ side is connected to [—Si(R)ₙL₃₋ₙ]. R⁸ is an alkyl group. Q³ is an alkylene group, a group having —C(O)NR⁶—, —C(O)—, —NR⁶— or —O— between carbon atoms of an alkylene group having two or more carbon atoms, or (OSi(R⁹)₂)ₚ—O—, and two or more Q³s may be the same as or different from each other. k3 is 2 or 3. R⁶ is a hydrogen atom, an alkyl group having 1 to 6 carbon atoms, or a phenyl group. R⁹ is an alkyl group, a phenyl group, or an alkoxy group, and two R⁹s may be the same as or different from each other. p is an integer from 0 to 5, and when p is two or more, the two or more (OSi(R⁹)₂) may be the same as or different from each other.

In the Formulas (g2-8) to (g2-14), the terminal bonded to CH₃(CH₂)ₘ₁— or —(CH₂)ₘ₂— is not —(CH₂)—.

The number of carbon atoms in the alkylene group of Q³ is preferably from 1 to 30, more preferably from 1 to 20, still more preferably from 2 to 20, and may be from 2 to 10 or from 2 to 6, from the viewpoint of ease of production of the compound and more preferable abrasion resistance of the surface treatment layer. Examples thereof include 2, 3, 8, 9, and 11. The number of carbon atoms may be from 1 to 10, from 1 to 6, or from 1 to 4. Here, the lower limit of the number of carbon atoms of the alkylene group in the case of having a specific bond between carbon atoms is 2.

The number of carbon atoms in the alkyl group of R⁸ is preferably from 1 to 6, more preferably from 1 to 3, and still more preferably from 1 to 2, from the viewpoint of ease of production of the compound.

The number of carbon atoms in the alkyl group of R⁹ is preferably from 1 to 6, more preferably from 1 to 3, and still more preferably from 1 to 2, from the viewpoint of ease of production of the compound.

The number of carbon atoms in the alkoxy group of R⁹ is preferably from I to 6, more preferably from 1 to 3, and still more preferably from 1 to 2, from the viewpoint of favorable storage stability of the compound.
p is preferably 0 or 1.

Examples of the Compound (3-1) and the Compound (3-2) include compounds of the following formulas. The compounds of the following formulas are preferable from the viewpoint of easy industrial production, easy handling, and more favorable abrasion resistance of the surface treatment layer. R^{c1} and R^{c2} in the compounds of the following formulas are the same as CH₃(CH₂)ₘ₁— in the Formula (3-1) and —(CH₂)ₘ₂— in the Formula (3-2) described above, respectively, and preferable embodiments are also the same.

Examples of the Compound (3-1) in which Y¹⁰ is a Group (g2-1) include compounds of the following formulas.

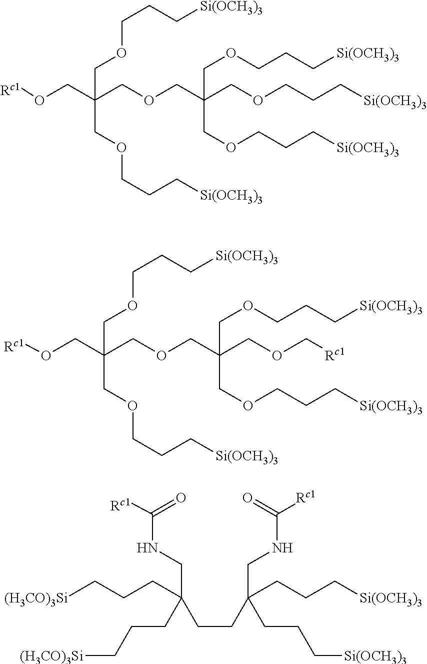

Examples of the Compound (3-1) in which Y¹⁰ is a Group (g2-2) include compounds of the following formulas.

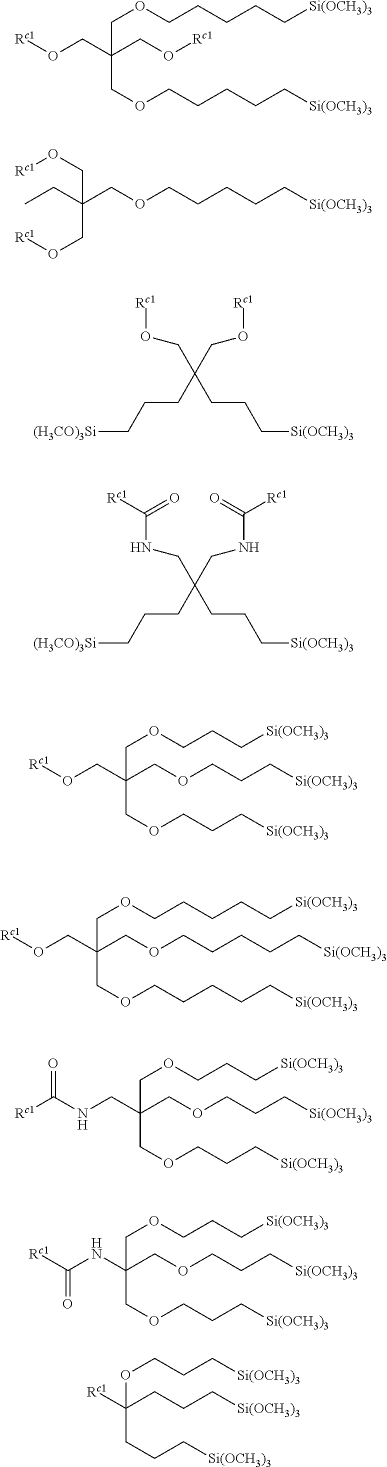

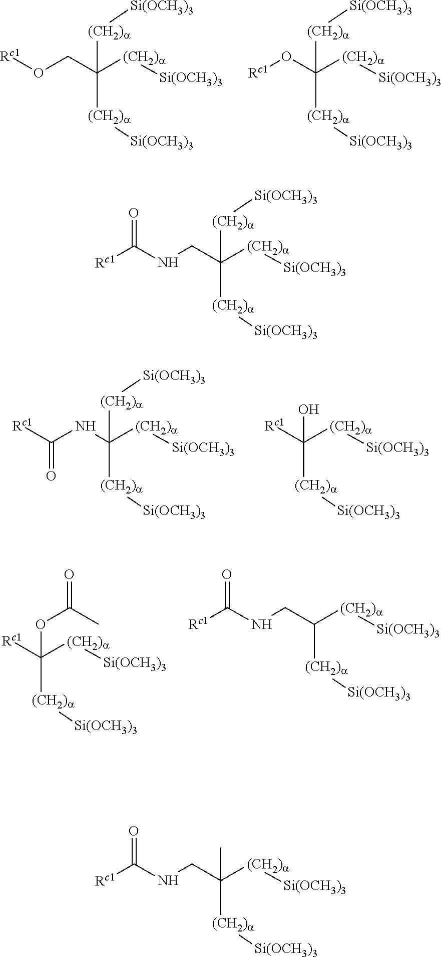

Examples of the Compound (3-1) in which Y¹⁰ is a Group (g2-3) include compounds of the following formulas.

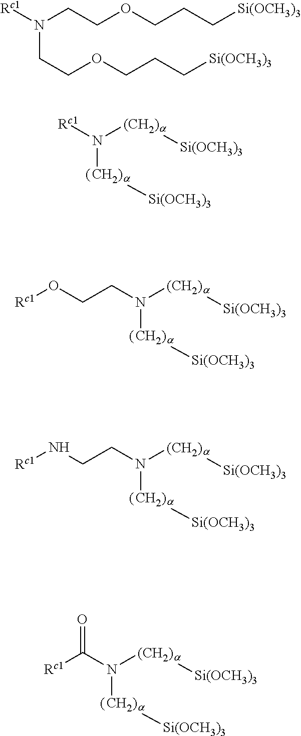

Examples of the Compound (3-1) in which Y¹⁰ is a Group (g2-4) include compounds of the following formulas.

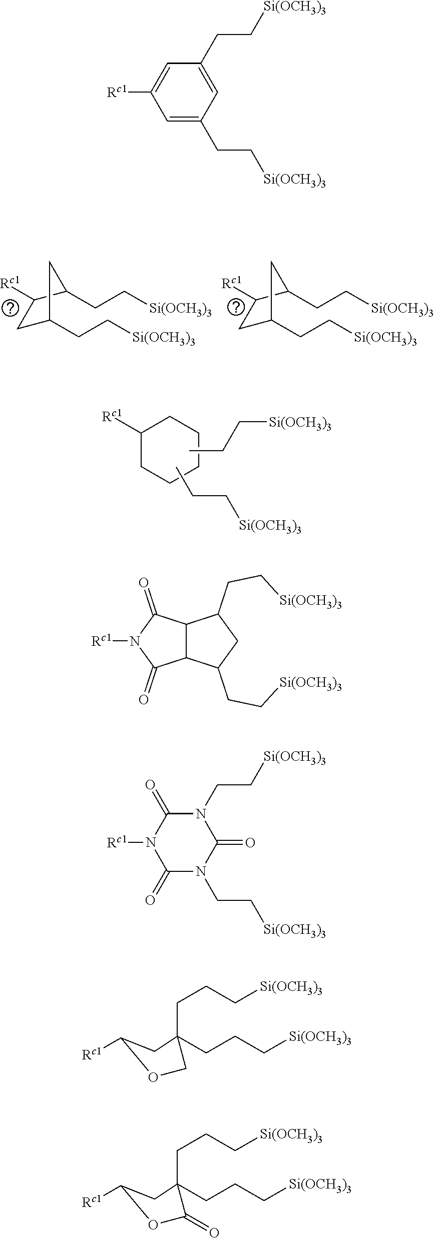

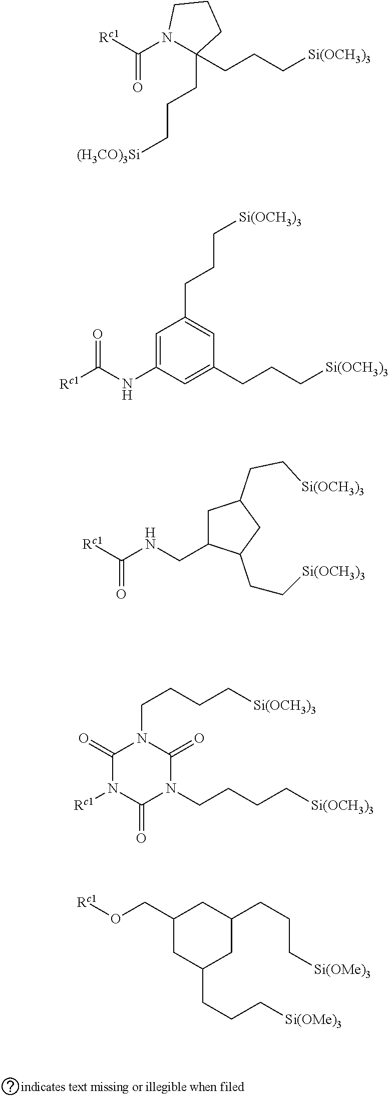

Examples of the Compound (3-1) in which Y¹⁰ is a Group (g2-5) include compounds of the following formulas.

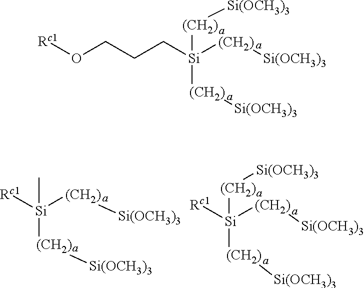

Examples of the Compound (3-1) in which Y¹⁰ is a Group (g2-6) include compounds of the following formulas.

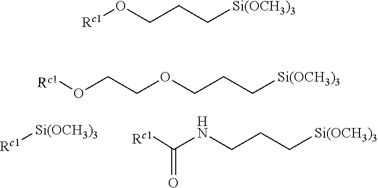

Examples of the Compound (3-1) in which Y¹⁰ is a Group (g2-7) include compounds of the following formulas.

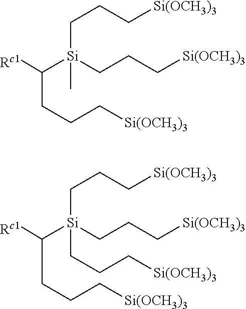

Examples of the Compound (3-1) in which Y¹⁰ is a Group (g2-8) include compounds of the following formulas.

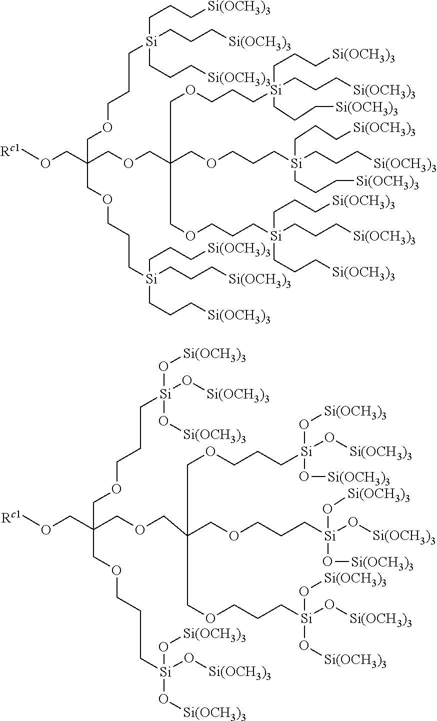

Examples of the Compound (3-1) in which Y¹⁰ is a Group (g2-9) include compounds of the following formulas.

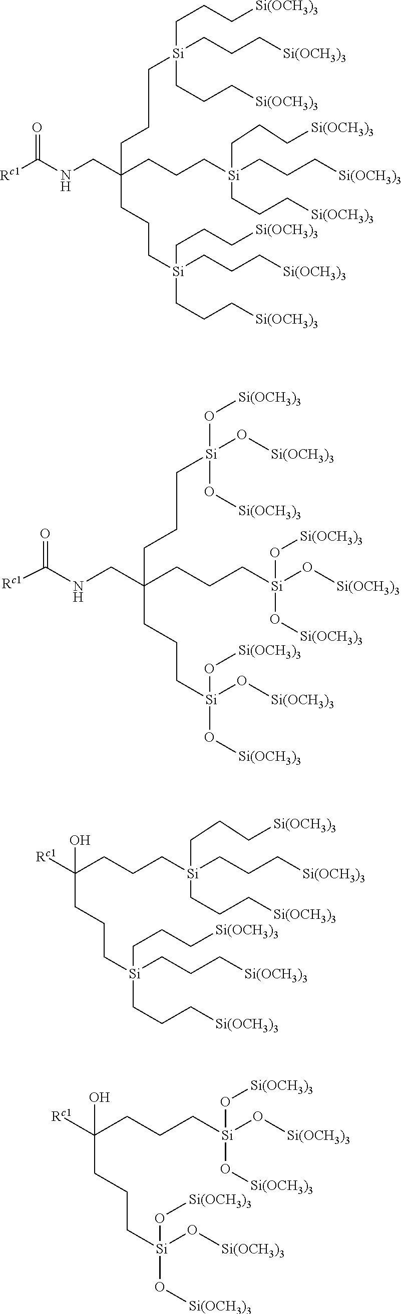

Examples of the Compound (3-1) in which Y¹⁰ is a Group (g2-10) include compounds of the following formulas.

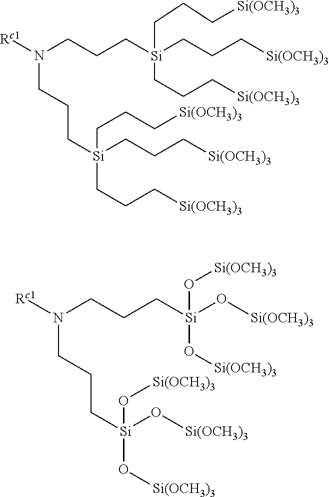

Examples of the Compound (3-1) in which Y¹⁰ is a Group (g2-11) include compounds of the following formulas.

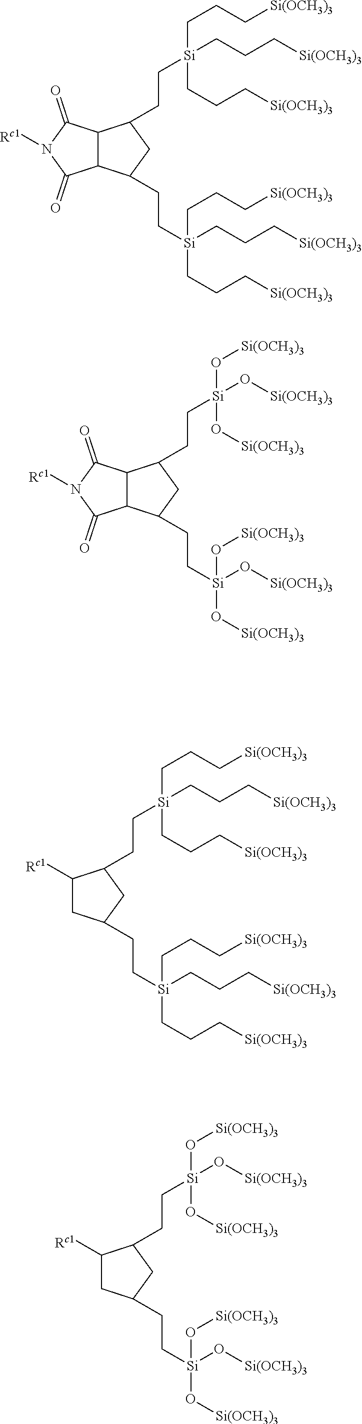

Examples of the Compound (3-1) in which Y¹⁰ is a Group (g2-12) include compounds of the following formulas.

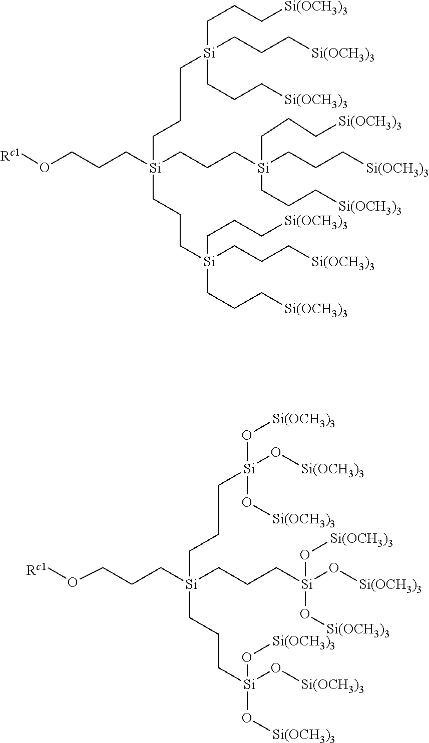

Examples of the Compound (3-1) in which Y¹⁰ is a Group (g2-13) include a compound of the following formula.

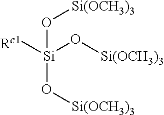

Examples of the Compound (3-1) in which Y¹⁰ is a Group (g2-14) include compounds of the following formulas.

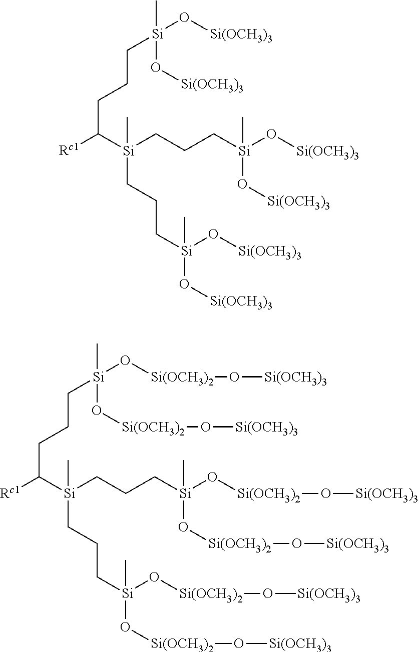

Examples of the Compound (3-2) in which each of Y¹¹ and Y¹² is a Group (g2-1) include a compound of the following formula.

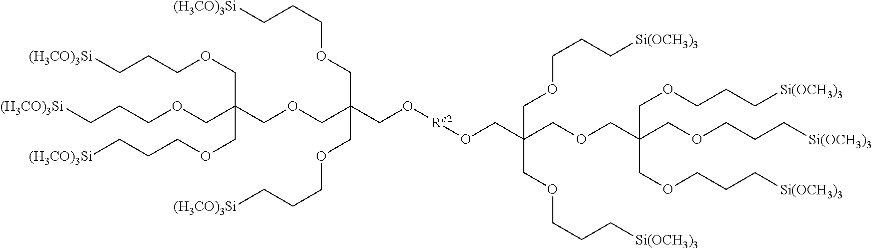

Examples of the Compound (3-2) in which each of Y¹¹ and Y¹² is a Group (g2-2) include compounds of the following formulas.

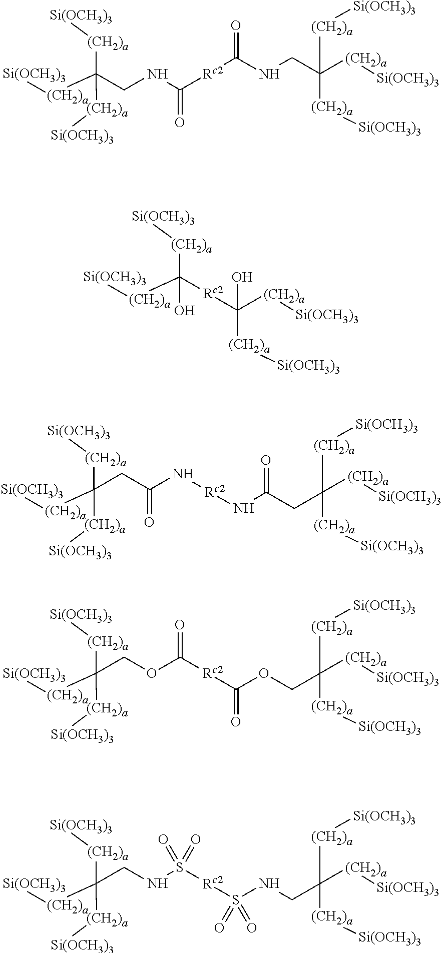

Examples of the Compound (3-2) in which each of Y¹¹ and Y¹² is a Group (g2-3) include a compound of the following formula.

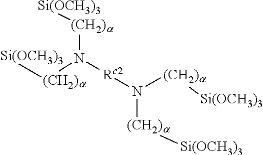

Examples of the Compound (3-2) in which each of Y¹¹ and Y¹² is a Group (g2-4) include a compound of the following formula.

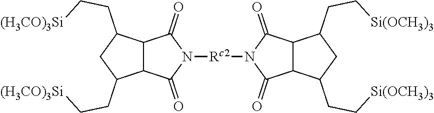

Examples of the Compound (3-2) in which each of Y¹¹ and Y¹² is a Group (g2-5) include a compound of the following formula.

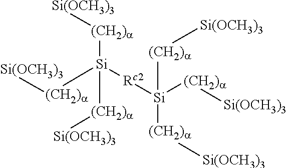

Examples of the Compound (3-2) in which each of Y¹¹ and Y¹² is a Group (g2-6) include a compound of the following formula.

(H₃CO)₃Si—R^{c2}—Si(OCH₃)₃

Examples of the Compound (3-2) in which each of Y¹¹ and Y¹² is a Group (g2-7) include a compound of the following formula.

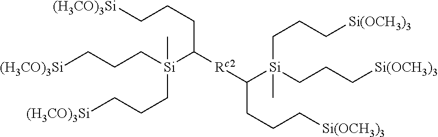

Examples of the Compound (3-2) in which each of Y¹¹ and Y¹² is a Group (g2-9) include compounds of the following formulas.

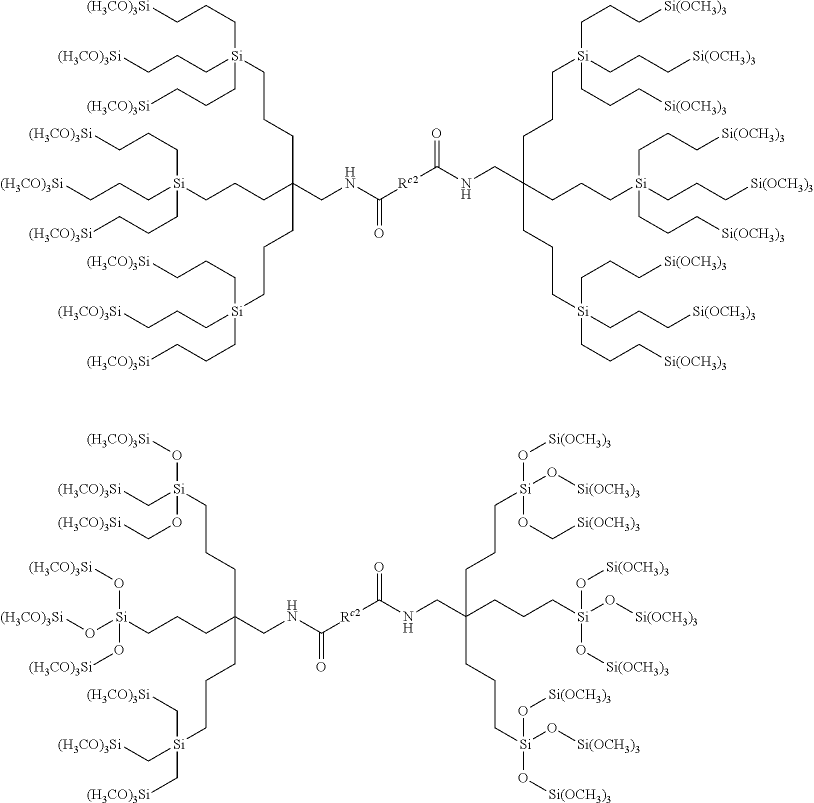

Examples of the compound according to the present disclosure include the following compounds.

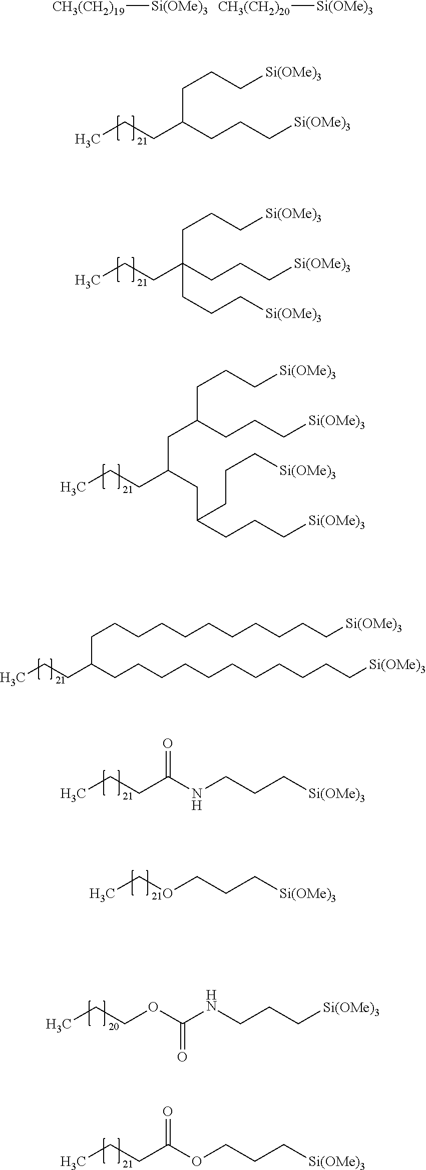

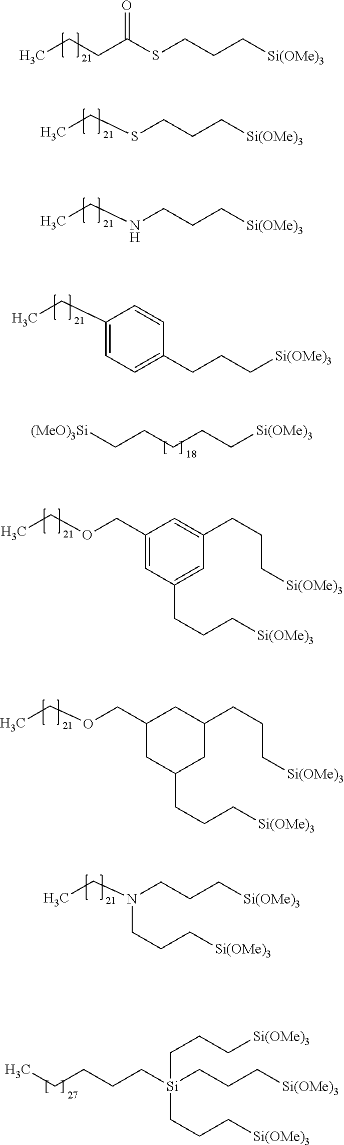

The number average molecular weight (Mn) of the compound according to the present disclosure is preferably from 400 to 20,000, more preferably from 500 to 18,000, and still more preferably from 600 to 15,000. When Mn is 400 or more, water repellency and abrasion resistance are excellent. When Mn is 20,000 or less, the viscosity is easily adjusted within an appropriate range, and the solubility is improved thereby improving the ease of handling during the film formation.

### [Method of Producing Compound]

The method of producing the compound according to the present disclosure is not particularly limited. Examples of the synthetic scheme for the compound according to the present disclosure are shown below.

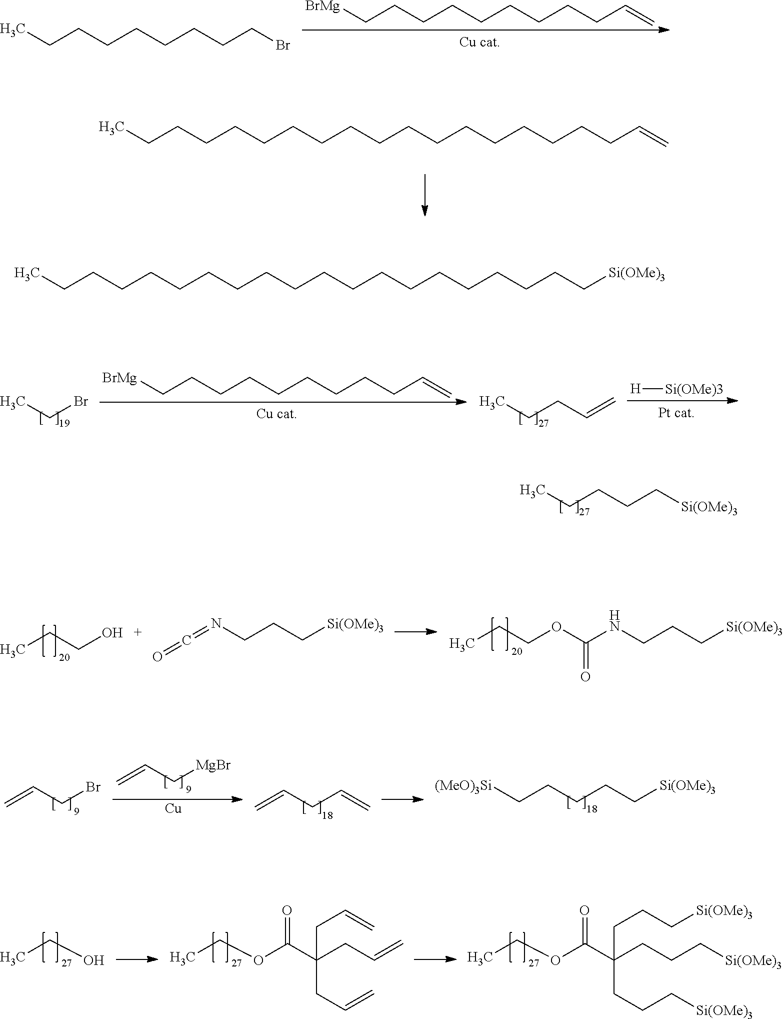

In one aspect, the compound according to the present disclosure is obtained by hydrosilylation of an intermediate represented by the following Formula (1-1) or (1-2).

[CH₃(CH₂)ₘ₁—]ⱼ₁Y^{10a}(B)_{g1} (1-1)

(B)ₖ₂Y^{12a}—(CH₂)ₘ₂—Y^{11a}(B)_{g2} (1-2)

In the Formula (1-1),
Y^{10a} is a single bond or a (j+g1)-valent linking group,
each B independently represents -Q^{L}-CH═CH₂, in which Q^{L} is a single bond or a divalent linking group,
m1 is an integer of 18 or more,
j1 is an integer of 1 or more, and
g1 is an integer of 1 or more, and
in the Formula (I-2),
Y^{11a} is a single bond or a (g2+1)-valent linking group,
Y^{12a} is a single bond or a (k2+1)-valent linking group,
each B independently represents -Q^{L}-CH═CH₂, in which Q^{L} is a single bond or a divalent linking group,
m2 is an integer of 19 or more,
k2 is an integer of 1 or more, and
g2 is an integer of 1 or more.
Y^{10a}(B)_{g}i in the Formula (1-1) is a compound in which, when the terminal in Y¹⁰[—Si(R)ₙL₃₋ₙ]_{g1} in the Formula (3-1) is a —CH₂—CH₂—Si(R)ₙL₃₋ₙ group, the —CH₂—CH₂—Si(R)ₙL₃₋ₙ group is replaced with —CH═CH₂.
Y^{11a}(B)_{g2} in the Formula (1-2) is a compound in which, when the terminal in Y¹¹[—Si(R)ₙL₃₋ₙ]_{g2} in the Formula (3-2) is a —CH₂—CH₂—Si(R)ₙL₃₋ₙ group, the —CH₂—CH₂—Si(R)ₙL₃₋ₙ group is replaced with —CH═CH₂.

Examples of the intermediate of the compound according to the present disclosure are shown below.

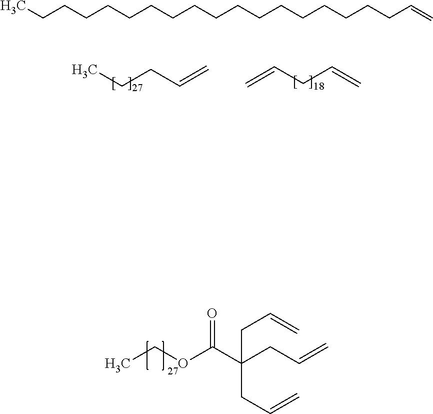

### [Composition]

The composition according to the present disclosure contains the compound according to the present disclosure, and components other than the compound according to the present disclosure are not particularly limited. The composition according to the present disclosure preferably contains a compound according to the present disclosure and a liquid medium. When the composition according to the present disclosure includes a liquid medium, the composition according to the present disclosure may be a solution, or may be a dispersion, as long as the composition is liquid.

The composition according to the present disclosure may contain impurities such as by-products generated in the production step of the compound according to the present disclosure, as long as the compound according to the present disclosure is contained.

The composition according to the present disclosure may contain one kind of the compound according to the present disclosure, or two or more kinds thereof.

The composition according to the present disclosure may contain either one of the single-ended compound or the double-ended compound, or may contain both. When the composition according to the present disclosure contains both the single-ended compound and the double-ended compound, the ratio of these two is not particularly limited, and the ratio of single-ended compound:double-ended compound is preferably from 1:99 to 99:1 by mass ratio. From the viewpoint of more favorable water repellency, the foregoing ratio is more preferably from 50:50 to 99:1, and still more preferably from 70:30 to 99:1. The composition according to the present disclosure preferably contains the single-ended compound, more preferably contains 70% by mass or more, still more preferably 80% by mass or more, of the single-ended compound with respect to the total amount of the compound according to the present disclosure.

The content rate of the compound according to the present disclosure is preferably from 0.001 to 40% by mass, more preferably from 0.01 to 20% by mass, and still more preferably from 0.1 to 10% by mass, with respect to the total amount of the composition according to the present disclosure. In the case of the composition according to the present disclosure used in a wet coating method, the content rate of the compound according to the present disclosure may be from 0.01 to 10% by mass, from 0.02 to 5% by mass, from 0.03 to 3% by mass, or from 0.05 to 2% by mass, with respect to the total amount of the composition according to the present disclosure.

The composition may contain one kind of liquid medium, or two or more kinds thereof.

The liquid medium is preferably an organic solvent.

Examples of the organic solvent include a compound composed only of a hydrogen atom and a carbon atom, and a compound composed only of a hydrogen atom, a carbon atom, and an oxygen atom, and specific examples thereof include a hydrocarbon-based organic solvent, a ketone-based organic solvent, an ether-based organic solvent, an ester-based organic solvent, a glycol-based organic solvent, and an alcohol-based organic solvent.

Specific examples of the hydrocarbon-based organic solvent include pentane, hexane, heptane, octane, hexadecane, isohexane, isooctane, isononane, cycloheptane, cyclohexane, bicyclohexyl, benzene, toluene, ethylbenzene, o-xylene, m-xylene, p-xylene, o-diethylbenzene, m-diethylbenzene, p-diethylbenzene, n-butylbenzene, sec-butylbenzene, and tert-butylbenzene.

Specific examples of the ketone-based organic solvent include acetone, methyl ethyl ketone, methyl isobutyl ketone, diisobutyl ketone, cyclohexanone, 2-heptanone, 4-heptanone, 3,5,5-trimethyl-2-cyclohexene-1-one, and 3,3,5-trimethylcyclohexanone, and isophorone.

Specific examples of the ether-based organic solvent include diethyl ether, cyclopentyl methyl ether, tetrahydrofuran, and 1,4-dioxane.

Specific examples of the ester-based organic solvent include methyl acetate, ethyl acetate, propyl acetate, isopropyl acetate, butyl acetate, isobutyl acetate, tert-butyl acetate, amyl acetate, isoamyl acetate, ethyl 3-ethoxypropionate, ethyl lactate ethylene glycol monobutyl ether acetate, diethylene glycol monobutyl ether acetate, propylene glycol monomethyl ether acetate, dipropylene glycol methyl ether acetate, 3-methoxy-3-methylbutyl acetate, 3-methoxybutyl acetate, propylene glycol monomethyl acetate, propylene glycol dimethyl acetate, ethylene glycol monoethyl ether acetate, ethylene glycol monomethyl ether acetate, diethylene glycol monoethyl ether acetate, cyclohexanol acetate, propylene glycol diacetate, propylene glycol monomethyl ether acetate, propylene glycol monomethyl ether propionate, propylene glycol monoethyl ether acetate, propylene glycol monobutyl ether acetate, propylene glycol monopropyl ether acetate, dipropylene glycol methyl ether acetate, 1,3-butylene glycol diacetate, 1,4-butanediol diacetate, 1,3-butylene glycol diacetate, 1,6-hexanediol diacetate, γ-butyrolactone, triacetin, and 2,2,4-trimethyl-1,3-pentanediol monoisobutyrate.

Specific examples of the glycol-based organic solvent include ethylene glycol, ethylene glycol monobutyl ether, diethylene glycol monobutyl ether, triethylene glycol monobutyl ether, tetraethylene glycol monobutyl ether, ethylene glycol monohexyl ether, diethylene glycol monohexyl ether, ethylene glycol mono-2-ethylhexyl ether, diethylene glycol mono-2-ethylhexyl ether, propylene glycol monomethyl ether, propylene glycol monoethyl ether, propylene glycol monobutyl ether, propylene glycol monopropyl ether, ethylene glycol monoisopropyl ether, ethylene glycol monoethyl ether, ethylene glycol mono-tert-butyl ether, ethylene glycol monopropyl ether, ethylene glycol monomethyl ether, diethylene glycol monoisopropyl ether, diethylene glycol monomethyl ether, dipropylene glycol monoethyl ether, dipropylene glycol monobutyl ether, dipropylene glycol monopropyl ether, dipropylene glycol monomethyl ether, tripropylene glycol monobutyl ether, tripropylene glycol monomethyl ether, propylene glycol monophenyl ether, 1,3-butylene glycol, propylene glycol n-propyl ether, propylene glycol n-butyl ether, diethylene glycol monoethyl ether, dipropylene glycol n-propyl ether, dipropylene glycol n-butyl ether, tripropylene glycol methyl ether, tripropylene glycol n-butyl ether, ethylene glycol dimethyl ether, ethylene glycol diethyl ether, ethylene glycol dibutyl ether, diethylene glycol diethyl ether, diethylene glycol dimethyl ether, dipropylene glycol dimethyl ether, diethylene glycol dibutyl ether, tetraethylene glycol dimethyl ether, dipropylene glycol dimethyl ether pentane, triethylene glycol dimethyl ether, and polyethylene glycol dimethyl ether.

Specific examples of the alcohol-based organic solvent include methanol, ethanol, 1-propanol, isopropyl alcohol, n-butanol, diacetone alcohol, isobutanol, sec-butanol, tert-butanol, pentanol, 3-methyl-1,3-butanediol, 1,3-butanediol, 1,3-butylene glycol, octanediol, 2,4-diethylpentanediol, butylethylpropanediol, 2-methyl-1,3-propanediol, 4-hydroxy-4-methyl-2-pentanone, 2-ethyl-1-hexanol, 3,5,5-trimethyl-1-hexanol, isodecanol, isotridecanol, 3-methoxy-3-methyl-1-butanol, 2-methoxybutanol, 3-methoxybutanol, cyclohexanol, furfuryl alcohol, tetrahydrofurfuryl alcohol, benzyl alcohol, and methylcyclohexanol.

Examples of the organic solvents include a halogen-based organic solvent, a nitrogen-containing compound, a sulfur-containing compound, and a siloxane compound.

Specific examples of the halogen-based organic solvent include dichloromethane, chloroform, carbon tetrachloride, dichloroethane, chlorobenzene, o-chlorotoluene, m-chlorotoluene, p-chlorotoluene, m-dichlorobenzene, and 1,2,3-trichloropropane.

Examples of the nitrogen-containing compound include nitrobenzene, acetonitrile, benzonitrile, N,N-dimethylformamide, N,N-dimethylacetamide, N-methylpyrrolidone, and 1,3-dimethyl-2-imidazolidinone.

Examples of the sulfur-containing compound include carbon disulfide and dimethyl sulfoxide.

Examples of the siloxane compound include hexamethyldisiloxane, octamethyltrisiloxane, and decamethyltetrasiloxane.

The content rate of the liquid medium is preferably from 60 to 99.999% by mass, more preferably from 80 to 99.99% by mass, and still more preferably from 90 to 99.9% by mass, with respect to the total amount of the composition according to the present disclosure. In the case of the composition according to the present disclosure used in a wet coating method, the content rate of the liquid medium may be from 90 to 99.99% by mass, from 95 to 99.98% by mass, from 97 to 99.97% by mass, or from 98 to 99.95% by mass, with respect to the total amount of the composition according to the present disclosure.

The composition according to the present disclosure may contain other components in addition to the compound according to the present disclosure and the liquid medium, as long as the effects of the present disclosure are not impaired.

Examples of said other components include known additives such as an acid catalyst and a basic catalyst that promote hydrolysis and condensation reaction of reactive silyl groups.

In addition, examples of said other components include a metal compound having a hydrolyzable group (hereinafter, the metal compound having a hydrolyzable group is also referred to as “specific metal compound”). When the composition according to the present disclosure contains a specific metal compound, slipping properties and antifouling properties of the surface treatment layer can be further improved. Examples of the specific metal compound include the following Formulas (M1) to (M3).

M(X^{b1})ₘ₁(X^{b2})ₘ₂(X^{b3})ₘ₃ (M1)

Si(X^{b4})(X^{b5})₃ (M2)

(X^{b6})₃Si—(Y^{b1})—Si(X^{b7})₃ (M3)

In the Formula (M1),
M represents a trivalent or tetravalent metal atom.
Each X^{b1} independently represents a hydrolyzable group.
Each X^{b2} independently represents a siloxane backbone-containing group.
Each X^{b3} independently represents a hydrocarbon chain-containing group.
m1 represents an integer from 2 to 4,
each of m2 and m3 independently represents an integer from 0 to 2, and
when M is a trivalent metal atom, m1+m2+m3 is 3, and when M is a tetravalent metal atom, m1+m2+m3 is 4.

In the Formula (M2),
X^{b4} represents a hydrolyzable silane oligomer residue.
Each X^{b5} independently represents a hydrolyzable group or an alkyl group having 1 to 4 carbon atoms.

In the Formula (M3),
Each of X^{b6} and X^{b7} independently represents a hydrolyzable group or a hydroxyl group.
Y^{b1} represents a divalent organic group.

In the Formula (M1), the metal represented by M also encompasses a metalloid such as Si or Ge. M is preferably a trivalent metal or a tetravalent metal, more preferably Al, Fe, In, Hf, Si, Ti, Sn, or Zr, still more preferably Al, Si, Ti, or Zr, and particularly preferably Si.

Examples of the hydrolyzable group represented by X^{b1} in the Formula (M1) include the same group as the hydrolyzable group represented by L in [—Si(R¹)ₙL₃₋ₙ] in the above-described reactive silyl group.

The siloxane backbone-containing group represented by X^{b2} has a siloxane unit (—Si—O—), and may be linear or branched. The siloxane unit is preferably a dialkylsilyloxy group, and examples thereof include a dimethylsilyloxy group and a diethylsilyloxy group. The number of repetitions of the siloxane unit in the siloxane backbone-containing group is 1 or more, preferably from 1 to 5, more preferably from 1 to 4, and still more preferably from 1 to 3.

The siloxane backbone-containing group may contain a divalent hydrocarbon group in a portion of the siloxane backbone. Specifically, some oxygen atoms of the siloxane backbone may be replaced by divalent hydrocarbon groups. Examples of the divalent hydrocarbon groups include alkylene groups such as a methylene group, an ethylene group, a propylene group, and a butylene group.

A hydrolyzable group, a hydrocarbon group (preferably an alkyl group), or the like may be bonded to the silicon atom at the terminal of the siloxane backbone-containing group.

The number of elements of the siloxane backbone-containing group is preferably 100 or less, more preferably 50 or less, still more preferably 30 or less. The number of elements is preferably 10 or more.

The siloxane backbone-containing group is preferably a group represented by *—(O—Si(CH₃)₂)ₙCH₃, in which n represents an integer from 1 to 5, and * represents a binding site for an adjacent atom.

The hydrocarbon chain-containing group represented by X^{b3} may be a group consisting only of a hydrocarbon chain, or may be a group having an etheric oxygen atom between carbon atoms of a hydrocarbon chain. The hydrocarbon chain may be linear or branched, and is preferably linear. The hydrocarbon chain may be a saturated hydrocarbon chain or an unsaturated hydrocarbon chain, and is preferably a saturated hydrocarbon chain. The number of carbon atoms in the hydrocarbon chain-containing group is preferably from 1 to 3, more preferably 1 or 2, and still more preferably 1. The hydrocarbon chain-containing group is preferably an alkyl group, and more preferably a methyl group, an ethyl group, or a propyl group.
m1 is preferably 3 or 4.

As the compound represented by the Formula (M1), compounds represented by the following Formulas (M1-1) to (M1-5) in which M is Si are preferable, and a compound represented by Formula (M1-1) is more preferable. The compound represented by Formula (M1-1) is preferably tetraethoxysilane, tetramethoxysilane, or triethoxymethylsilane.

Si(X^{b1})₄ (M1-1)

CH₃—Si(X^{b1})₃ (M1-2)

C₂H₅—Si(X^{b1})₃ (M1-3)

n—C₃H₇—Si(X^{b1})₃ (M1-4)

(CH₃)₂CH—Si(X^{b1})₃ (M1-5)

In the Formula (M2), the number of silicon atoms contained in the hydrolyzable silane oligomer residue represented by X^{b4} is preferably 3 or more, more preferably 5 or more, still more preferably 7 or more. The number of the silicon atoms is preferably 15 or less, more preferably 13 or less, still more preferably 10 or less.

The hydrolyzable silane oligomer residue may have an alkoxy group bonded to a silicon atom. Examples of the alkoxy group include a methoxy group, an ethoxy group, a propoxy group, and a butoxy group, and a methoxy group and an ethoxy group are preferable. The hydrolyzable silane oligomer residue may have one kind or two or more kinds of these alkoxy groups, and preferably has one kind thereof.

Examples of the hydrolyzable silane oligomer residue include (C₂H₅O)₃Si—(OSi(OC₂H₅)₂)₄O—*. Here, * represents a bonding position to an adjacent atom.

Examples of the hydrolyzable group represented by X^{b5} in the Formula (M2) include the same group as the hydrolyzable group represented by L in [—Si(R′)ₙL₃₋ₙ] in the above-described reactive silyl group, a cyano group, a hydrogen atom, and an allyl group, and an alkoxy group or an isocyanato group is preferable. The alkoxy group is preferably an alkoxy group having 1 to 4 carbon atoms.
X^{b5} is preferably a hydrolyzable group.

Examples of the compound represented by the Formula (M2) include (H₅C₂O)₃—Si—(OSi(OC₂H₅)₂)₄OC₂H₅.

The compound represented by the Formula (M3) is a compound having reactive silyl groups at both terminals of a divalent organic group, that is, bissilane.

Examples of the hydrolyzable group represented by X^{b6} and X^{b7} in the Formula (M3) include an alkoxy group, an acyloxy group, a ketoxime group, an alkenyloxy group, an amino group, an aminoxy group, an amide group, an isocyanato group, and a halogen atom, and an alkoxy group and an isocyanato group are preferable. As the alkoxy group, an alkoxy group having 1 to 4 carbon atoms is preferable, and a methoxy group and an ethoxy group are more preferable.

In the Formula (M3), X^{b6} and X^{b7} may be the same group or different groups. From the viewpoint of availability, X^{b6} and X^{b7} are preferably the same group.

In the Formula (M3), Y^{b1} is a divalent organic group linking the reactive silyl groups at both terminals. The number of carbon atoms in the divalent organic group Y^{b1} is preferably from 1 to 8, and more preferably from 1 to 3.

Examples of Y^{b1} include an alkylene group, a phenylene group, and an alkylene group having an etheric oxygen atom between carbon atoms. Examples thereof include —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(CH₃)₂CH₂—, —C(CH₃)₂CH₂CH₂C(CH₃)₂—, —CH₂CH₂OCH₂CH₂—, —CH₂CH₂CH₂OCH₂CH₂CH₂—, —CH(CH₃)CH₂OCH₂CH(CH₃)—, and —C₆H₄—.

Examples of the compound represented by the Formula (M3) include (CH₃O)₃Si(CH₂)₂Si(OCH₃)₃, (C₂H₅O)₃Si(CH₂)₂Si(OC₂H₅)₃, (OCN)₃Si(CH₂)₂Si(NCO)₃, Cl₃Si(CH₂)₂SiCl₃, (CH₃₀)₃Si(CH₂)₆Si(OCH₃)₃, and (C₂H₅O)₃Si(CH₂)₆Si(OC₂H₅)₃.

The content rate of said other components that may be contained in the composition according to the present disclosure is preferably 10% by mass or less, and more preferably 1% by mass or less, with respect to the total amount of the composition according to the present disclosure. When the composition according to the present disclosure contains the specific metal compound, the content rate of the specific metal compound is preferably from 0.01 to 30% by mass, more preferably from 0.01 to 10% by mass, and still more preferably 0.05 to 5% by mass, with respect to the total amount of the composition according to the present disclosure.

The total content rate of the compound according to the present disclosure and said other components (hereinafter, also referred to as “solid content concentration”) is preferably from 0.001 to 40% by mass, more preferably from 0.01 to 20% by mass, and still more preferably from 0.1 to 10% by mass, with respect to the total amount of the composition according to the present disclosure. The solid content concentration of the composition according to the present disclosure is a value calculated from the mass of the composition before heating and the mass after heating in a convection dryer at 120° C. for 4 hours.

The composition according to the present disclosure contains a liquid medium, and therefore, it is useful for coating applications and can be used as a coating liquid.

### [Surface Treatment Agent]

In one aspect, the surface treatment agent according to the present disclosure contains a compound according to the present disclosure. In addition, the surface treatment agent according to the present disclosure may contain the compound according to the present disclosure and a liquid medium. The surface treatment agent according to the present disclosure may be the composition according to the present disclosure. Preferred embodiments of the liquid medium contained in the surface treatment agent are the same as the preferred embodiments of the liquid medium contained in the composition according to the present disclosure.

Since the compound according to the present disclosure has the foregoing configuration, use of a surface treatment agent containing the compound according to the present disclosure allows for forming a surface treatment layer having excellent water repellency and abrasion resistance.

### [Article]

In one aspect, the article according to the present disclosure includes a substrate and a surface treatment layer formed by surface treatment with the surface treatment agent according the present disclosure.

The surface treatment layer may be formed on a portion of the surface of the substrate, or may be formed on the entire surface of the substrate. The surface treatment layer may spread in a film shape on the surface of the substrate, or may be scattered in a dot shape.

In the surface treatment layer, the compound according to the present disclosure is contained in a state in which a part or all of the reactive silyl groups have undergone hydrolysis, and in which the dehydration condensation reaction of the silanol groups has progressed.

The thickness of the surface treatment layer is preferably from 1 to 100 nm, and more preferably from 1 to 50 nm. When the thickness of the surface treatment layer is 1 nm or more, the effect of the surface treatment tends to be sufficiently obtained. When the thickness of the surface treatment layer is 100 nm or less, utilization efficiency is high. The thickness of the surface treatment layer can be calculated by obtaining an interference pattern of reflected X-ray by X-ray reflectivity using an X-ray diffractometer for thin film analysis (product name “ATX-G”, manufactured by RIGAKU Corporation), and calculating the thickness from the vibration period of the interference pattern.

The type of the substrate is not particularly limited, and examples thereof include a substrate for which impartation of water repellency is desired. Examples of the substrate include: a substrate that may be used by contacting other articles (e.g., stylus) or human fingers; a substrate that may be held by human fingers during operation; and a substrate that may be placed on another article (e.g., a mounting table).

Examples of the material of the substrate include metal, resin, glass, sapphire, ceramic, stone, fiber, nonwoven fabric, paper, wood, natural leather, artificial leather, and composite materials thereof. The glass may be chemically strengthened.

Examples of the substrate include a building material, a decorative building material, interior goods, transportation equipment (e.g., an automobile), a signboard, a bulletin board, a drinking vessel, tableware, a water tank, an ornamental instrument (e.g., a frame, a box), a laboratory instrument, furniture, a textile product, and a packaging container; glass or resin used for art, sports, games, or the like; and glass or resin used for exterior portions (excluding a display unit) in a device such as a mobile phone (e.g., a smartphone), a portable information terminal, a game machine, or a remote controller. The shape of the substrate may be a plate shape or a film shape.

As the substrate, a substrate for a touch panel, a substrate for a display, and a spectacle lens are suitable, and a substrate for a touch panel is particularly suitable. The material of the substrate for a touch panel is preferably glass or a transparent resin.

The substrate may be a substrate whose one or both surfaces have been subjected to a surface treatment, such as corona discharge treatment, plasma treatment, or plasma graft polymerization treatment. The substrate that has been subjected to surface treatment has better adhesion to the surface treatment layer, and the abrasion resistance of the surface treatment layer is further improved. Therefore, it is preferable to perform the surface treatment on the surface of the substrate that contacts the surface treatment layer. In a case in which an underlayer described later is provided, the substrate that has been subjected to the surface treatment has more favorable adhesion to the underlayer, and the abrasion resistance of the surface treatment layer is further improved. Therefore, when the underlayer is provided, it is preferable to perform the surface treatment on the surface of the substrate that contacts the underlayer.

The surface treatment layer may be provided directly on the surface of the substrate, or the underlayer may be provided between the substrate and the surface treatment layer. From the viewpoint of further improving the water repellency and abrasion resistance of the surface treatment layer, the article according to the present disclosure preferably includes a substrate, an underlayer disposed on the substrate, and a surface treatment layer disposed on the underlayer and formed by surface-treatment with the surface treatment agent according to the present disclosure.

The underlayer is preferably a layer containing an oxide containing silicon and at least one specific element selected from the group consisting of Group 1 elements, Group 2 elements, Group 4 elements, Group 5 elements, Group 13 elements, and Group 15 elements of the periodic table.

The Group 1 elements of the periodic table (hereinafter, also referred to as “Group 1 elements”) refer to lithium, sodium, potassium, rubidium, and cesium. As Group 1 elements, lithium, sodium, and potassium are preferable, and sodium and potassium are more preferable, from the viewpoint that the surface treatment layer can be more uniformly formed on the underlayer without defects, or the variation in the composition of the underlayer between samples is further suppressed. The underlayer may contain two or more kinds of Group 1 elements.

The Group 2 elements of the periodic table (hereinafter, also referred to as “Group 2 elements”) refer to beryllium, magnesium, calcium, strontium, and barium. As Group 2 elements, magnesium, calcium, and barium are preferable, and magnesium and calcium are more preferable, from the viewpoint that the surface treatment layer can be more uniformly formed on the underlayer without defects, or the variation in the composition of the underlayer between samples is further suppressed. The underlayer may contain two or more kinds of Group 2 elements.

The Group 4 elements of the periodic table (hereinafter, also referred to as “Group 4 elements”) refer to titanium, zirconium, and hafnium. As Group 4 elements, titanium and zirconium are preferable, and titanium is more preferable, from the viewpoint that the surface treatment layer can be more uniformly formed on the underlayer without defects, or from the viewpoint that variations in the composition of the underlayer between samples are further suppressed. The underlayer may contain two or more kinds of Group 4 elements.

The Group 5 elements of the periodic table (hereinafter, also referred to as “Group 5 elements”) refer to vanadium, niobium, and tantalum. As Group 5 elements, vanadium is particularly preferable from the viewpoint of more favorable abrasion resistance of the surface treatment layer. The underlayer may contain two or more kinds of Group 5 elements.

The Group 13 elements of the periodic table (hereinafter, also referred to as “Group 13 elements”) refer to boron, aluminum, gallium, and indium. As Group 13 elements, boron, aluminum, and gallium are preferable, and boron and aluminum are more preferable, from the viewpoint that the surface treatment layer can be more uniformly formed on the underlayer without defects or the variation in the composition of the underlayer between samples is further suppressed. The underlayer may contain two or more kinds of Group 13 elements.

The Group 15 elements of the periodic table (hereinafter, also referred to as “Group 15 elements”) refer to nitrogen, phosphorus, arsenic, antimony, and bismuth. As Group 15 elements, phosphorus, antimony, and bismuth are preferable, and phosphorus and bismuth are more preferable, from the viewpoint that the surface treatment layer can be more uniformly formed on the underlayer without defects, or from the viewpoint that variation in the composition of the underlayer between samples is further suppressed. The underlayer may contain two or more kinds of Group 15 elements.

As the specific element contained in the underlayer, Group 1 elements, Group 2 elements, and Group 13 elements are preferable since the abrasion resistance of the surface treatment layer is more favorable, and Group 1 elements and Group 2 elements are more preferable, and Group 1 elements are still more preferable.

As the specific element, only one kind of element may be contained, or two or more kinds of elements may be contained.

The oxide contained in the underlayer may be a mixture of simple oxides of the foregoing elements (silicon and the specific element) (e.g., a mixture of a silicon oxide and an oxide of the specific element), a composite oxide containing two or more kinds of the foregoing elements, or a mixture of a simple oxide of any of the foregoing elements and a composite oxide.

The ratio of the total molar concentration of the specific element in the underlayer to the molar concentration of silicon in the underlayer (specific element/silicon) is preferably from 0.02 to 2.90, more preferably from 0.10 to 2.00, and still more preferably from 0.20 to 1.80, from the viewpoint of more favorable abrasion resistance of the surface treatment layer.

The molar concentration (mol %) of each element in the underlayer can be measured by, for example, depth direction analysis by X-ray photoelectron spectroscopy (XPS) using ion sputtering.

The underlayer may be a single layer or a multilayer. The underlayer may have an uneven surface.

The thickness of the underlayer is preferably from 1 to 100 nm, more preferably from 1 to 50 nm, and still more preferably from 2 to 20 nm. When the thickness of the underlayer is equal to or greater than the foregoing lower limit, the adhesion of the underlayer to the surface treatment layer is further improved, and the abrasion resistance of the surface treatment layer is further improved. When the thickness of the underlayer is equal to or less than the foregoing upper limit, the underlayer itself has excellent abrasion resistance.

The thickness of the underlayer is measured by observing a cross-section of the underlayer with a transmission electron microscope (TEM).

The underlayer can be formed by, for example, a vapor deposition method using a vapor deposition material, or a wet coating method.

The vapor deposition material used in the vapor deposition method preferably contains silicon and an oxide containing the specific element.

Specific examples of the form of the vapor deposition material include a powder, a molten body, a sintered body, a granulated body, and a crushed body, and from the viewpoint of ease of handling, a molten body, a sintered body, and a granulated body are preferable.

The molten body is a solid obtained by melting a powder of the vapor deposition material at a high temperature and then cooling and solidifying the powder. The sintered body is a solid obtained by firing a powder of the vapor deposition material, and a molded body obtained by press-molding the powder may be used instead of the powder of the vapor deposition material as necessary. The granulated body is a solid obtained by kneading a powder of the vapor deposition material and a liquid medium (e.g., water or an organic solvent) to form particles and then drying the particles.

The deposition material may be produced, for example, by the following method.
A method of mixing a powder of silicon oxide and a powder of an oxide of the specific element to obtain a powder of the vapor deposition material.
A method of kneading the powder of the vapor deposition material and water to form particles, and then drying the particles to form a granulated body of the vapor deposition material.
A method of drying a mixture obtained by mixing a powder containing silicon (for example, a powder made of silicon oxide, silica sand, or silica gel), a powder containing the specific element (e.g., a powder of an oxide of the specific element, a carbonate, a sulfate, a nitrate, an oxalate, or a hydroxide), and water, and then firing the dried mixture or a molded body obtained by press-molding the dried-mixture to obtain a sintered body.
A method of melting, at a high temperature, a powder containing silicon (e.g., a powder made of silicon oxide, silica sand, or silica gel) and a powder containing the specific element (e.g., a powder of an oxide of the specific element, a carbonate, a sulfate, a nitrate, an oxalate, or a hydroxide), and then cooling and solidifying the melt to obtain a molten body.

Specific examples of the vapor deposition method using the vapor deposition material include a vacuum vapor deposition method. The vacuum vapor deposition method is a method of evaporating a vapor deposition material in a vacuum chamber and attaching the material to the surface of a substrate.

The temperature during the vapor deposition (for example, in a case in which a vacuum vapor deposition apparatus is used, the temperature of the boat on which the vapor deposition material is disposed) is preferably from 100 to 3,000° C., and more preferably from 500 to 3,000° C.

The pressure (for example, in a case in which a vacuum vapor deposition apparatus is used, the pressure in a tank in which the vapor deposition material is disposed) during the vapor deposition is preferably 1 Pa or less, and more preferably 0.1 Pa or less.

When the underlayer is formed using the vapor deposition material, one vapor deposition material may be used, or two or more kinds of vapor deposition materials containing different elements may be used.

Specific examples of the evaporation method of the vapor deposition material include a resistance heating method of melting and evaporating the vapor deposition material on a resistance heating boat made of high melting point metal, and an electron gun method of irradiating the vapor deposition material with an electron beam and directly heating the vapor deposition material to melt the surface and evaporate the vapor deposition material. As the method for evaporating the vapor deposition material, the electron gun method is preferable from the viewpoint that a high melting point substance can also be evaporated since it can be heated locally, and from the viewpoint that there is no risk of reaction with the container or contamination with impurities since the area not irradiated by the electron beam is at a low temperature.

As the evaporation method of a vapor deposition material, multiple boats may be used, or all vapor deposition materials may be placed in a single boat and used. The vapor deposition method may be co-vapor deposition, alternate vapor deposition, or the like. Specific examples thereof include an example in which silica and a specific source are mixed in the same boat and used, an example in which silica and a specific element source are placed in separate boats and co-deposited, and an example in which silica and a specific element source are similarly placed in separate boats and alternately deposited. Conditions, order, and the like of the vapor deposition are appropriately selected depending on the configuration of the underlayer.

In the wet coating method, it is preferable to form an underlayer on the substrate by a wet coating method using a coating liquid containing a compound containing silicon, a compound containing the specific element, and a liquid medium.

Specific examples of the silicon compound include silicon oxide, silicic acid, and partial condensates of silicic acid, alkoxysilane, and partial hydrolysis condensates of alkoxysilane.

Specific examples of the compound containing the specific element include an oxide of the specific element, an alkoxide of the specific element, a carbonate of the specific element, a sulfate of the specific element, a nitrate of the specific element, an oxalate of the specific element, and a hydroxide of the specific element.

Examples of the liquid medium include the same liquid mediums as those contained in the composition according to the present disclosure.

The content rate of the liquid medium is preferably from 0.01 to 20% by mass, and more preferably from 0.1 to 10% by mass, with respect to the total amount of the coating liquid used for forming the underlayer.

Specific examples of the wet coating method of forming the underlayer include a spin coating method, a wipe coating method, a spray coating method, a squeegee coating method, a dip coating method, a die coating method, an inkjet method, a flow coating method, a roll coating method, a casting method, a Langmuir-Blodgett method, and a gravure coating method.

After the wet-coating with the coating liquid, it is preferable to dry the coating film. The temperature for drying the coating film is preferably from 20 to 200° C., and more preferably from 80 to 160° C.

The article according to the present disclosure is preferably an optical member. Examples of the optical member include a car navigation, a mobile phone, a smartphone, a digital camera, a digital video camera, a PDA, a portable audio player, a car audio, a game machine, a spectacle lens, a camera lens, a lens filter, sunglasses, a medical instrument such as a gastroscope, a copying machine, a PC, a display (e.g., a liquid crystal display, an organic EL display, a plasma display, or a touch panel display), a touch panel, a protective film, and an antireflection film. In particular, the article is preferably a display or a touch panel.

### [Method of Producing Article]

The method of producing an article according to the present disclosure is, for example, a method of performing a surface treatment on a substrate using the surface treatment agent according to the present disclosure to produce an article having a surface treatment layer formed on the substrate. Examples of the surface treatment include a dry coating method and a wet coating method.

Examples of the dry coating method include methods such as vacuum deposition, CVD, and sputtering. As the dry coating method, a vacuum vapor deposition method is preferable from the viewpoint of suppressing decomposition of the compounds and convenience of the apparatus. At the time of vacuum deposition, a pelletized material obtained by impregnating a metal porous body of iron, steel, or the like with the compound according to the present disclosure may be used. A pelletized material impregnated with the compound according to the present disclosure, obtained by impregnating a metal porous body of iron, steel or the like with a composition containing the compound according to the present disclosure and a liquid medium and drying the liquid medium, may also be used.

Examples of the wet coating method include a spin coating method, a wipe coating method, a spray coating method, a squeegee coating method, a dip coating method, a die coating method, an inkjet method, a flow coating method, a roll coating method, a casting method, a Langmuir-Blodgett method, and a gravure coating method.

In order to improve the abrasion resistance of the surface treatment layer, an operation for promoting the reaction between the compound according to the present disclosure and the substrate may be performed as necessary. Examples of the operation include heating, humidification, and light irradiation.

For example, a substrate on which the surface treatment layer is formed may be heated in the atmosphere containing moisture to promote reactions such as hydrolysis reaction of hydrolyzable groups, reaction between silanol groups and hydroxy groups or the like on the surface of the substrate, and generation of siloxane bonds by condensation reaction of silanol groups.

After the surface treatment, compounds in the surface treatment layer that are not chemically bonded to another compound or the substrate may be removed as necessary. Examples of the removal method include a method of pouring a solvent over the surface treatment layer and a method of wiping the surface treatment layer with a cloth soaked with a solvent.

### EXAMPLES

Hereinafter, embodiments of the present disclosure will be specifically described with reference to Examples. However, the embodiments of the present disclosure are not limited to these Examples.

### Synthesis Example 1: Synthesis of Compound (1A)

1-Bromononane (2.0 g), tetrahydrofuran (THF) (30 mL), 0.5 M undeca-10-enylmagnesium bromide in THE (30 mL), and copper chloride (0.1 g) were added and stirred at 50° C. for 24 hours. Hydrochloric acid and dichloromethane were added thereto, and extraction was performed. The solvent was distilled off, and then flash column chromatography was performed using silica gel (developing solvent: ethyl acetate/hexane) to obtain 1.7 g of Compound (1A).

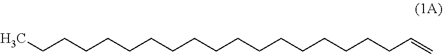 [SMILES: C=CCCCCCCCCCCCCCCCCCC]

¹H NMR (500 MHz, Chloroform-d) δ 5.80 (tt, J=13.4, 6.1 Hz, 1H), 5.05 (dddt, J=78.0, 13.4, 2.0, 1.0 Hz, 2H), 2.14-1.94 (m, 2H), 1.47-1.31 (m, 2H), 1.31-1.19 (m, 30H), 0.98-0.77 (m, 3H).

### Synthesis Example 2: Synthesis of Compound (1B)

1,3-Bistrifluoromethylbenzene (10 g) was added to the Compound (1A) (1.7 g), and the mixture was stirred. A toluene solution of platinum/1,3-divinyl-1,1,3,3-tetramethyldisiloxane complex (platinum content rate 3%, 0.1 g), aniline (0.1 g), and trimethoxysilane (1.0 g) were added thereto, and the mixture was stirred at 40° C. for 24 hours. Thereafter, the solvent was distilled off under reduced pressure to obtain 2.5 g of Compound (1B).

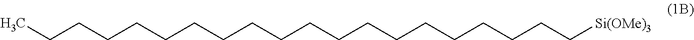

¹H NMR (500 MHz, Chloroform-d) δ 3.58 (s, 9H), 1.39 (pd, J=7.0, 1.0 Hz, 2H), 1.33-1.20 (m, 34H), 0.97-0.82 (m, 3H), 0.62 (t, J=7.0 Hz, 2H).

### Synthesis Example 3: Synthesis of Compound (2A)

THE (30 mL), 0.5M undeca-10-enylmagnesium bromide in THE (30 mL), and copper chloride (0.1 g) were added to 1-bromoeicosane (3.6 g), and the mixture was stirred at 50° C. for 24 hours. Hydrochloric acid and dichloromethane were added thereto, and extraction was performed. The solvent was distilled off, and then flash column chromatography was performed using silica gel (developing solvent: ethyl acetate/hexane) to obtain 2.5 g of Compound (2A).

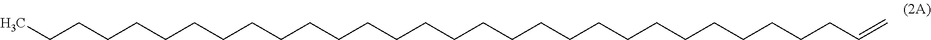 [SMILES: C=CCCCCCCCCCCCCCCCCCCCCCCCCCCCCC]

¹H NMR (500 MHz, Chloroform-d) δ 5.80 (tt, J=13.6, 6.1 Hz, 1H), 5.05 (dddt, J=77.9, 13.6, 2.4, 1.1 Hz, 2H), 2.10-1.96 (m, 2H), 1.52-1.18 (m, 54H), 0.98-0.75 (m, 3H).

### Synthesis Example 4: Synthesis of Compound (2B)

1,3-Bistrifluoromethylbenzene (10 g) was added to the Compound (2A) (2.5 g) and the mixture was stirred. A toluene solution of platinum/1,3-divinyl-1,1,3,3-tetramethyldisiloxane complex (platinum content rate 3%, 0.1 g), aniline (0.1 g), and trimethoxysilane (1.0 g) were added and stirred at 40° C. for 24 hours. Thereafter, the solvent was distilled off under reduced pressure to obtain 2.8 g of Compound (2B).

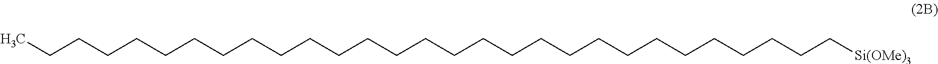

¹H NMR (500 MHz, Chloroform-d) δ 3.58 (s, 9H), 1.54-1.20 (m, 58H), 1.00-0.83 (m, 3H), 0.62 (t, J=7.0 Hz, 2H).

### Synthesis Example 5: Synthesis of Compound (3A)

(3-Isocyanatopropyl)trimethoxysilane (2.1 g) and THE (10 g) were added to 1-docosanol (3.3 g) and stirred at 70° C. for 24 hours. After cooling to 25° C., the solvent was distilled off under reduced pressure to obtain 4.9 g of Compound (3A).

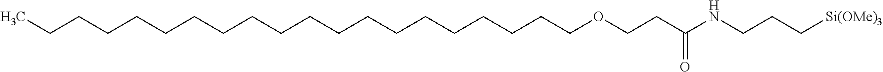

¹H NMR (500 MHz, Chloroform-d) δ 4.85 (t, J=7.0 Hz, 1H), 4.18-4.00 (m, 2H), 3.58 (s, 9H), 3.13 (q, J=7.1 Hz, 2H), 1.68 (dp, J=26.7, 7.1 Hz, 4H), 1.43-1.17 (m, 38H), 0.96-0.82 (m, 3H), 0.68 (t, J=7.1 Hz, 2H).

### Synthesis Example 6: Synthesis of Compound (4A)

Compound (4A) was obtained according to the method described in Synthesis Examples 3 and 4 of WO 2021/054413 A1.

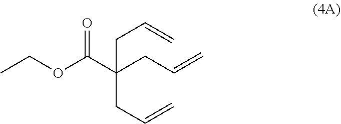

### Synthesis Example 7: Synthesis of Compound (4B)

Water (50 g) and potassium hydroxide (5 g) were added to the Compound (4A) (10 g), and the mixture was heated and refluxed at 65° C. for 24 hours. After cooling to 25° C., hydrochloric acid and dichloromethane were added, and extraction was performed. The solvent was distilled off, and then flash column chromatography was performed using silica gel (developing solvent: methanol/dichloromethane) to obtain 8.3 g of Compound (4B).

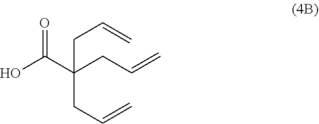

¹H NMR (500 MHz, Chloroform-d) δ 5.71 (ddt, J=13.7, 12.6, 6.2 Hz, 3H), 5.16 (dt, J=13.4, 1.1 Hz, 6H), 2.20 (dt, J=6.2, 0.9 Hz, 6H).

### Synthesis Example 8: Synthesis of Compound (4C)

The Compound (4B) (2.0 g) and THE (10 mL) were added to 1-octacosanol (4.1 g), and the mixture was stirred, and then, N-methylmorpholine (2.0 g) and 4-(4,6-dimethoxy-1,3,5-triazine-2-yl)-4-methylmorpholinium chloride (4.0 g) were added thereto. The temperature was raised to 50° C., and the mixture was stirred for 24 hours. After cooling to 25° C., flash column chromatography was performed using silica gel (developing solvent: ethyl acetate/hexane) to obtain 3.5 g of Compound (4C).

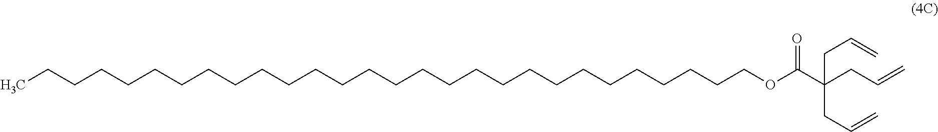 [SMILES: C=CCC(CC=C)(CC=C)C(=O)OCCCCCCCCCCCCCCCCCCCCCCCCCCCC]

¹H NMR (500 MHz, Chloroform-d) δ 5.81-5.56 (m, 3H), 5.16 (dt, J=13.4, 1.1 Hz, 6H), 4.07 (t, J=7.1 Hz, 2H), 2.22 (dt, J=6.2, 1.1 Hz, 6H), 1.67 (p, J=7.1 Hz, 2H), 1.47-1.14 (m, 50H), 0.98-0.78 (m, 3H).

### Synthesis Example 9: Synthesis of Compound (4D)

1,3-Bistrifluoromethylbenzene (10 g) was added to the Compound (4C) (3.0 g) and the mixture was stirred. A toluene solution of platinum/1,3-divinyl-1,1,3,3-tetramethyldisiloxane complex (platinum content rate 3%, 0.1 g), aniline (0.1 g), and trimethoxysilane (1.0 g) were added thereto, and the mixture was stirred at 40° C. for 24 hours. Thereafter, the solvent was distilled off under reduced pressure to provide 3.4 g of Compound (4D).

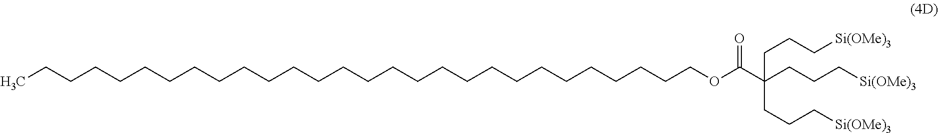

¹H NMR (500 MHz, Chloroform-d) δ 4.07 (t, J=7.1 Hz, 2H), 3.58 (s, 27H), 1.78-1.43 (m, 14H), 1.42-1.19 (m, 52H), 0.99-0.81 (m, 3H), 0.72 (t, J=7.0 Hz, 6H).

### Synthesis Example 10: Synthesis of Compound (5A)

THE (30 mL), 0.5M undeca-10-enylmagnesium bromide in THE (30 mL), and copper chloride (0.1 g) were added to 11-bromo-1-undecene (2.4 g), and the mixture was stirred at 50° C. for 24 hours. Hydrochloric acid and dichloromethane were added thereto, and extraction was performed. The solvent was distilled off, and then flash column chromatography was performed using silica gel (developing solvent: ethyl acetate/hexane) to obtain 2.0 g of Compound (5A).

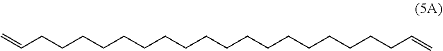 [SMILES: C=CCCCCCCCCCCCCCCCCCCC=C]

¹H NMR (500 MHz, Chloroform-d) δ 5.80 (tt, J=13.4, 6.1 Hz, 2H), 5.05 (dddt, J=78.0, 13.4, 2.1, 0.9 Hz, 4H), 2.03 (tdt, J=7.2, 6.2, 1.0 Hz, 4H), 1.38 (pd, J=7.0, 0.7 Hz, 4H), 1.33-1.13 (m, 28H).

### Synthesis Example 11: Synthesis of Compound (5B)

1,3-Bistrifluoromethylbenzene (10 g) was added to the Compound (5A) (2.0 g), and the mixture was stirred. A toluene solution of platinum/1,3-divinyl-1,1,3,3-tetramethyldisiloxane complex (platinum content rate 3%, 0.1 g), aniline (0.1 g), and trimethoxysilane (1.0 g) were added thereto, and the mixture was stirred at 40° C. for 24 hours. Thereafter, the solvent was distilled off under reduced pressure to obtain 2.6 g of Compound (5B).

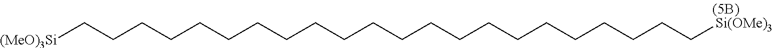

¹H NMR (500 MHz, Chloroform-d) δ 3.58 (s, 18H), 1.48-1.17 (m, 40H), 0.62 (t, J=7.0 Hz, 4H).

### [Production of Articles and Evaluation]

Substrates were surface-treated using each of the compounds obtained in Synthesis Examples 2, 4, 5, 9, and 11, as well as octadecyltrimethoxysilane (Tokyo Chemical Industry Co., Ltd., product code: 00256), to obtain articles of Examples 1 to 6. In each Example, the dry coating method described below was used as the surface treatment method. As the substrate, chemically strengthened glass was used. The obtained articles were evaluated by the following method. The results are shown in Table 1.

### (Dry Coating Method)

Dry coating was performed using a vacuum deposition apparatus (VTR350M manufactured by ULVAC, Inc.) (vacuum vapor deposition method). A molybdenum boat in the vacuum vapor deposition apparatus was filled with 0.5 g of a 20% ethyl acetate solution of each compound, and the inside of the vacuum vapor deposition apparatus was evacuated to 1×10⁻³ Pa or less. The boat in which the compound was placed was heated at a temperature increase rate of 10° C./min or less, and when the vapor deposition rate measured by the quartz crystal microbalance exceeded 1 nm/sec, the shutter was opened to initiate the film formation on the surface of the substrate. When the film thickness reached about 50 nm, the shutter was closed to finish the film formation on the surface of the substrate. The substrate on which the compound was deposited was heat-treated at 100° C. for 30 minutes to obtain an article having a surface treatment layer on the surface of the substrate.

### (Evaluation Method)

### <Water Repellency>

The contact angle of about 2 μL of distilled water placed on the surface of the surface treatment layer was measured using a contact angle measuring device (DM-500 manufactured by Kyowa Interface Science Co., Ltd.). Measurement was performed at five different positions on the surface of the surface treatment layer, and the average value thereof was calculated. The contact angle was calculated by a 20 method. The evaluation criteria are as follows. A is a level at which there is no problem in practical use.

### Initial Water Contact Angle:

A: 108 degrees or more.
B: less than 108 degrees.

### <Abrasion Resistance (Steel Wool)>

In accordance with JIS L0849:2013 (ISO 105-X12:2001), a steel wool bonster (#0000) was reciprocated on the surface treatment layer of each article 10,000 times at a pressure of 98.07 kPa and a speed of 320 cm/min using a reciprocating traverse tester (manufactured by KNT Co., Ltd.), and then the water contact angle was measured by the above-described method. The smaller the decrease in the water repellency (water contact angle) after abrasion is, the smaller the decrease in performance due to the friction is, and the more favorable the abrasion resistance is. The evaluation criteria are as follows.
AAA: Change in water contact angle after 10,000 reciprocations is 5 degrees or less.
AA: Change in water contact angle after 10,000 reciprocations is from more than 5 degrees to 8 degrees or less.
A: Change in water contact angle after 10,000 reciprocations is from more than 8 degrees to 10 degrees or less.
B: Change in water contact angle after 10,000 reciprocations is more than 10 degrees.

TABLE 1

| | 1 | 2 | 3 | 4 | 5 | |
|---|---|---|---|---|---|---|
| Example | Compound | Compound | Compound | Compound | Compound | 6 |
| Compound | (1B) | (2B) | (3A) | (4D) | (5B) | Octadecyltrimethoxysilane |
| Water | A | A | A | A | A | B |
| repellency | | | | | | |
| Abrasion | AA | AAA | AAA | AAA | AAA | A |
| resistance | | | | | | |

In the above examples, Examples 1 to 5 are working examples, and Example 6 is a comparative example. As shown in Table 1, Examples 1 to 5 achieved favorable evaluations of both water repellency and abrasion resistance, whereas Example 6 was unable to achieve both favorable water repellency and abrasion resistance.

### INDUSTRIAL APPLICABILITY

The compound according to the present disclosure is useful as a surface treatment agent. The surface treatment agent can be used, for example, for substrates in display devices such as touch panel displays, optical elements, semiconductor elements, building materials, automobile components, in nanoimprint technology, or the like. In addition, the surface treatment agent can be used for a body, a window glass (windshield, side glass, rear glass), a mirror, a bumper, and the like of transportation equipment such as trains, automobiles, ships, and airplanes. Furthermore, the surface treatment agent can be used for outdoor articles such as building exterior walls, tents, solar power generation modules, sound insulating plates, or concrete; fishing nets, insect trap nets, and water tanks. In addition, the surface treatment agent can be used for various indoor equipment such as: kitchens, bathrooms, wash basins, mirrors, and toilet-related components; ceramics such as chandeliers and tiles; artificial marble and air conditioners. In addition, the surface treatment agent can be used for antifouling treatment for jigs, inner walls, pipes, and the like in factories. In addition, the surface treatment agent can be used for goggles, glasses, helmets, pachinko, fibers, umbrellas, playing tools, and soccer balls.

In addition, the surface treatment agent can be used as an adhesion inhibitor for various packaging materials, such as food packaging materials, cosmetic packaging materials, and the inside of pots. In addition, the surface treatment agent can be used for optical members such as car navigations, mobile phones, smartphones, digital cameras, digital video cameras, PDAs, portable audio players, car audios, game machines, spectacle lenses, camera lenses, lens filters, sunglasses, and medical instruments such as gastroscopes, copying machines, PCs, displays (e.g., liquid crystal displays, organic EL displays, plasma displays, or touch panel displays), touch panels, protective films, and antireflection films.

The disclosure of Japanese Patent Application No. 2022-049074 filed on Mar. 24, 2022 is incorporated herein by reference in its entirety. Furthermore, all documents, patent applications, and technical standards described in the present specification are incorporated herein by reference to the same extent as if each individual document, patent application, and technical standard were specifically and individually stated to be incorporated by reference.

1. A compound comprising: a linear alkyl group having 19 or more carbon atoms or a linear alkylene group having 19 or more carbon atoms; and a reactive silyl group linked to the linear alkyl group having 19 or more carbon atoms or the linear alkylene group having 19 or more carbon atoms, directly or via a polyvalent linking group.

2. The compound according to claim 1, wherein a number of carbon atoms of the linear alkyl group having 19 or more carbon atoms or the linear alkylene group having 19 or more carbon atoms is from 19 to 30.

3. The compound according to claim 1, wherein the reactive silyl group comprises an alkoxysilyl group.

4. A compound represented by the following Formula (1):
(A)ⱼY¹[—Si(R)ₙL₃₋ₙ]_{g} (1)
wherein, in the Formula (1),
A is CH₃(CH₂)ₘ₁— or [L₃₋ₙ(R)ₙSi—]ₖY²—(CH₂)ₘ₂—, wherein m1 is an integer of 18 or more, m2 is an integer of 19 or more, Y² is a single bond or a (k+1)-valent linking group, k is an integer of 1 or more, and definitions of R, L, and n are the same as definitions of R, L, and n in the Formula (1), respectively,
Y¹ is a single bond or a (j+g)-valent linking group,
each R independently represents a monovalent hydrocarbon group,
each L independently represents a hydrolyzable group or a hydroxyl group,
n is an integer from 0 to 2,
j is an integer of 1 or more, and
g is an integer of 1 or more.

5. The compound according to claim 4, wherein, in the Formula (1), m1 is from 18 to 29 and m2 is from 19 to 30.

6. The compound according to claim 4, wherein at least one L is an alkoxy group in the Formula (1).

7. The compound according to claim 4, wherein:
in the Formula (1),
A is CH₃(CH₂)ₘ₁—, and g is an integer of 2 or more, or
A is [L₃₋ₙ(R)ₙSi—]ₖY²—(CH₂)ₘ₂—, and each of g and k is independently an integer of 2 or more.

8. A compound represented by the following Formula (1-1) or (1-2):
[CH₃(CH₂)ₘ₁—]ⱼ₁Y^{10a}(B)_{g1} (1-1)
(B)ₖ₂Y^{12a}—(CH₂)ₘ₂—Y^{11a}(B)_{g2} (1-2)
wherein:
in the Formula (1-1),
Y^{10a} is a single bond or a (j1+g1)-valent linking group,
each B independently represents -Q^{L}-CH═CH₂, wherein Q^{L} is a single bond or a divalent linking group,
m1 is an integer of 18 or more,
j1 is an integer of 1 or more, and
g1 is an integer of 1 or more,
in the Formula (1-2),
Y^{11a} is a single bond or a (g2+1)-valent linking group,
Y^{12a} is a single bond or a (k2+1)-valent linking group,
each B independently represents -Q^{L}-CH═CH₂, wherein Q^{L} is a single bond or a divalent linking group,
m2 is an integer of 19 or more,
k2 is an integer of 1 or more, and
g2 is an integer of 1 or more.

9. A composition comprising the compound according to claim 1 and a liquid medium.

10. A surface treatment agent comprising the compound according to claim 1.

11. The surface treatment agent according to claim 10, further comprising a liquid medium.

12. A method of producing an article, the method comprising: subjecting a substrate to a surface treatment with the surface treatment agent according to claim 10 to produce an article having a surface treatment layer formed on the substrate.

13. An article comprising: a substrate; and a surface treatment layer disposed on the substrate and formed by surface treatment with the surface treatment agent according to claim 10.

14. The article according to claim 13, wherein the article is an optical member.

15. The article according to claim 13, wherein the article is a display or a touch panel.
